(19) United States
(12) Patent Application Publication
(10) Pub. No.: US 2008/0058616 A1
(43) Pub. Date: Mar. 6, 2008

(54) Vital information measuring device, electric power consumption control method, vital information measuring program, and computer-readable storage medium

(75) Inventors: Katsuya Nakagawa, Kizugawa-shi (JP); Nobuyoshi Ishino, Uji-shi (JP)

Correspondence Address: BIRCH STEWART KOLASCH & BIRCH, PO BOX 747, FALLS CHURCH, VA, 22040-0747, US

(73) Assignee: SHARP KABUSHIKI KAISHA

(21) Appl. No.: 11/896,269

(22) Filed: Aug. 30, 2007

(30) Foreign Application Priority Data
Sep. 4, 2006 (JP) 2006-239557

(51) Int. Cl. A61B 5/00

(52) U.S. Cl. 600/301

(57) ABSTRACT

A vital information measuring device according to the present invention includes: communication means for communicating with an external device; a vital information measuring section for measuring vital information of a living organism; vital condition judging section for judging a condition of the living organism by comparing (i) the vital information measured by the vital information measuring section with (ii) vital evaluation information for evaluating the vital information; an electric power consumption control section for controlling electric power consumption of the vital information measuring device in accordance with a result of the judgment carried out by the vital condition judging means. This makes it possible to control the electric power consumption of the device in accordance with the result of judging the condition of the living organism.

This Nonprovisional application claims priority under 35 U.S.C. § 119(a) on Patent Application No. 2006/239557 filed in Japan on Sep. 4, 2006, the entire contents of which are hereby incorporated by reference.

### FIELD OF THE INVENTION

The present invention relates to (i) a vital information measuring device for measuring vital information and transmitting it via wireless communication or wired communication, (ii) a method for controlling electric power consumption of the vital information measuring device, (iii) an operation program for the vital information measuring device, and (iv) a computer-readable storage medium storing the operation program.

### BACKGROUND OF THE INVENTION

Conventionally, there has been used a vital information measuring device, which is put on a living organism so as to continuously measure vital information of the living organism in daily activity and transmits the measurement data in a wireless or wired manner for the purpose of managing the living organism's health condition.

Required for such continuous measurement of the vital information of the living organism in daily activity and constant management of the living organism's health condition is a vital information measuring device that is capable of continuously measuring the vital information without preventing the living organism's daily life as much as possible.

For realization of such a vital information measuring device, the vital information measuring device needs to be driven by a battery and needs to be downsized and have light weight. For attaining these, it is desirable to reduce electric power consumption of the vital information measuring device as much as possible.

Further, it is preferable that the vital information measuring device be capable of carrying out measurement at a short cycle or high frequency as much as possible so as to detect changes in the living organism's health condition at an early stage as much as possible and of transmitting the measurement data wirelessly. Also in the case of transmitting it in a wired manner, downsizing, weight saving, and reduction of electric power consumption of the vital information measuring device are still required.

Now, consider the case of the wireless communication, for example. Each of a telemetry ring type pulse oximeter AD7010 (hereinafter, simply referred to as “AD7010”) and a telemetry ring type pulse oximeter AD7011 (hereinafter, simply referred to as “AD7011”) provided by Advanced Medical Inc. is put on a finger so as to measure oxygen saturation in blood and pulse rate, and automatically transmits the measurement data every set time by using a radio compliant with the weak radio telemeter method.

Generally speaking, wireless communication consumes a lot of electric power. Although electric power consumption differs depending on a wireless communication method, electric power of approximately 120 mW is generally consumed in the case of Bluetooth®, whereas electric power of approximately 50 mW is required in the case of Zigbee® or the specified low power radio method.

For such frequent wireless transmission of the measurement data, a buttery having a capacity as large as possible is required. This is contradictory to the downsizing and weight saving of the vital information measuring device. If a battery having a small capacity is adopted in favor of the downsizing and weight saving of the vital information measuring device, it is necessary to frequently replace or charge the battery because such a battery has a short battery life.

A conceivable way to restrain electric consumption is to carry out wireless communication less frequently. However, this makes it difficult to detect changes in the living organism's health condition at an early stage. Accordingly, when there occurs something abnormal in the living organism's health condition, it is impossible to make a prompt action thereto, with the result that the living organism is likely to face problems serious to the health condition and life.

For example, the telemetry ring type pulse oximeter AD7010 provided by the Advanced Medical Inc. transmits measurement data every 6 hours, whereas the telemetry ring type pulse oximeter AD7011 transmits measurement data every 10 minutes or every 1 minute.

Hence, the AD7010 secures its battery life for approximately one month at maximum. However, because the AD7010 transmits measurement data every 6 hours, it is very difficult for the receiving end system to detect the living organism's abnormality at an early stage. On the other hand, the AD7011 transmits measurement data every 10 minutes or every 1 minute, so that it is possible for the receiving end system to detect the living organism's abnormality at an early stage; however, its battery life lasts approximately 1 week at maximum. Therefore, batteries need to be changed frequently, and the AD7011 thus suffers from a problem in practical use. Further, such frequent change of batteries imposes financial loads.

An example of conventional techniques for solving these problems is an implant radio relay system 100 disclosed in Patent Document 1: Japanese Unexamined Patent Publication Tokukai 2004-48361 (Feb. 12, 2004).

FIG. 8 is a schematic diagram illustrating the configuration of the conventional implant radio relay system 100. As shown in FIG. 8, the implant radio relay system 100 includes a radio device 101 implanted in a living organism, a radio relay device 102, and an external communication system 103. Here, the radio device 101 is connected to an implanted device that is not shown in FIG. 8. The radio relay device 102 relays wireless communication between the radio device 101 and the communication system 103, and is brought by a user.

Explained next are operations of the implant radio relay system 100. When the radio relay device 102 detects data generated by the implanted device (not shown) connected to the radio device 101 in the implant radio relay system 100, transmission electric power of the radio device 101 is appropriately controlled for the sake of reducing wasting of a battery of the radio device 101.

In the meanwhile, an alternative example of the conventional techniques is a health management system 200 disclosed in Patent Document 2: Japanese Unexamined Patent Publication Tokukai 2002-288347 (published on Oct. 4, 2002). FIG. 9 is a schematic diagram illustrating the configuration of the conventional health management system 200. As shown in FIG. 9, the health management system 200 is configured such that a sensor section 202 is connected to a mobile phone main body 201. The mobile phone main body 201 includes a monitoring control function section 203. Connected to the monitoring control function section 203 are an external interface section 204 and a mobile phone section 205.

The external interface section 204 is connected to an external device (not shown) such as a personal computer, which stores necessary information in advance such as (i) data threshold values for judging abnormality in human organism information regarding an examinee's health, (ii) a fixed contact time, and (iii) the telephone numbers of contact persons/places such as a hospital.

In the health management system 200, the sensor section 202 measures the human organism information regarding the examinee's health, and the monitoring control function section 203 makes reference to the information stored in advance in the external device (not shown), such as a personal computer, connected to the external interface section 204. When the monitoring control function section 203 judges, in accordance with the information thus referred, that the measurement data obtained from the sensor section 202 is abnormal and that the abnormality needs to be reported urgently, the monitoring control function section 203 causes the mobile phone section 205 to make an urgent report to a destination set in advance. This allows a third person to recognize the sudden change in the user's health in daily life.

However, the implant radio relay system 100 shown in FIG. 8 does not take into consideration for reduction of electric power consumption by controlling transmission electric power or transmission timing in accordance with the condition of the living organism (abnormality, emergency). Further, the implant radio relay system 100 assumes that the user always brings the radio relay device 102 with him/her. This imposes loads on the user's daily activity, and the entire system is complicated.

On the other hand, the health management system 200 of FIG. 9 does not provide any technique of reducing consumption of electric power required for the constant measurement carried out by the sensor section 202 connected to the mobile phone main body 201. First of all, the mobile phone main body 201 consumes a lot of electric power in general and runs out of battery in several days, so that the mobile phone main body 201 is not suitable for the constant measurement for vital information.

### SUMMARY OF THE INVENTION

An object of the present invention is to provide (i) a vital information measuring device that is capable of controlling its electric power consumption in accordance with a result of judging a condition of a living organism, and (ii) an electric power consumption control method.

To achieve the object, a vital information measuring device according to the present invention includes: communication means for communicating with an external device; measuring means for measuring vital information of a living organism; vital condition judging means for judging a condition of the living organism by comparing (i) the vital information measured by the measuring means with (ii) vital evaluation information for evaluating the vital information; and electric power consumption control means for controlling electric power consumption of the vital information measuring device in accordance with a result of the judgment carried out by the vital condition judging means.

According to the above configuration, the measuring means measures the vital information, and the communication means transmits the measured vital information to the external device via communication. The wording “communication” herein encompasses both wireless communication and wired communication. Further, the vital condition judging means compares the vital information measured by the measuring means with the vital evaluation information for evaluating the vital information, so as to judge the condition of the living organism. Further, the electric power consumption control means controls the electric power consumption of the vital information measuring device in accordance with the result of the judgment carried out by the vital condition judging means.

With this, the electric power consumption of the vital information measuring device of the present invention is controlled by the electric power consumption control means in accordance with the result of the judgment carried out by the vital condition judging means.

Here, the “vital evaluation information” may be any information allowing for evaluation of the condition of the living organism, such as (i) an “evaluation threshold value”, which is a threshold value for evaluating the condition of the living organism, and (ii) the combination of the “evaluation threshold value” and “vital reference information”, which is referred for more accurate judgment.

Further, the phrase “judging the vital condition of the living organism” means judging whether or not the vital information measured by the vital information measuring means indicates a value representing a normal condition of the living organism (whether or not the living organism has abnormality). For example, the judgment as to the condition of the living organism is a judgment as to whether or not there is abnormality or a sign of abnormality in vital information representing the living organism's circulatory system condition, such as pulse rate, blood pressure, or oxygen saturation concentration in blood.

To achieve the object, an electric power consumption control method according to the present invention includes: a measuring step in which measuring means of a vital information measuring device measures vital information of a living organism; a vital condition judging step in which vital condition judging means of the vital information measuring device judges a condition of the living organism by comparing (i) the vital information measured in the measuring step with (ii) vital evaluation information for evaluating the vital information; and an electric power consumption control step in which electric power consumption control means of the vital information measuring device controls electric power consumption of the vital information measuring device in accordance with a result of the judgment carried out in the vital condition judging step.

According to the above electric power consumption control method for the vital information measuring device of the present invention, it is possible to control the electric power consumption of the vital information measuring device in the electric power consumption control step in accordance with the judgment carried out in the vital condition judging step.

Additional objects, features, and strengths of the present invention will be made clear by the description below. Further, the advantages of the present invention will be evident from the following explanation in reference to the drawings.

### BRIEF DESCRIPTION OF THE DRAWINGS

FIG. 1 is a function block diagram illustrating one embodiment of a vital information measuring device of the present invention.

FIG. 2 is a flowchart illustrating operations of the vital information measuring device.

FIG. 3(A) is a table illustrating examples of evaluation threshold values for pulse rate, which evaluation threshold values are used in the vital information measuring device.

FIG. 3(B) is a table illustrating examples of evaluation threshold values for blood pressure, which evaluation threshold values are used in the vital information measuring device.

FIG. 3(C) is a table illustrating examples of evaluation threshold values of oxygen saturation in arterial blood, which evaluation threshold values are used in the vital information measuring device.

FIG. 3(D) is a table illustrating examples of evaluation threshold values for body temperature, which evaluation threshold values are used in the vital information measuring device.

FIG. 4(A) is a table illustrating basic information, which is an example of vital reference information used by the vital information measuring device.

FIG. 4(B) is a table illustrating physical information, which is an example of the vital reference information used by the vital information measuring device.

FIG. 4(C) is a table illustrating medical history-health information, which is an example of the vital reference information used by the vital information measuring device.

FIG. 5(A) is a table illustrating examples of measurement values of normal pulse rate measured by the vital information measuring device.

FIG. 5(B) is a table illustrating examples of measurement values of abnormal pulse rate measured by the vital information measuring device.

FIG. 6(A) is a schematic diagram illustrating an example of presentation made to a living organism (user) when the measurement data obtained by the vital information measuring device indicates a normal value.

FIG. 6(B) is a schematic diagram illustrating an example of presentation made to the living organism (user) when the measurement data obtained by the vital information measuring device indicates an abnormal value.

FIG. 7 is a schematic diagram illustrating an example regarding the living organism's (user's) operation on the vital information measuring device.

FIG. 8 is a schematic diagram illustrating the configuration of a conventional implant radio relay system.

FIG. 9 is a schematic diagram illustrating the configuration of a conventional health management system.

### DESCRIPTION OF THE EMBODIMENTS

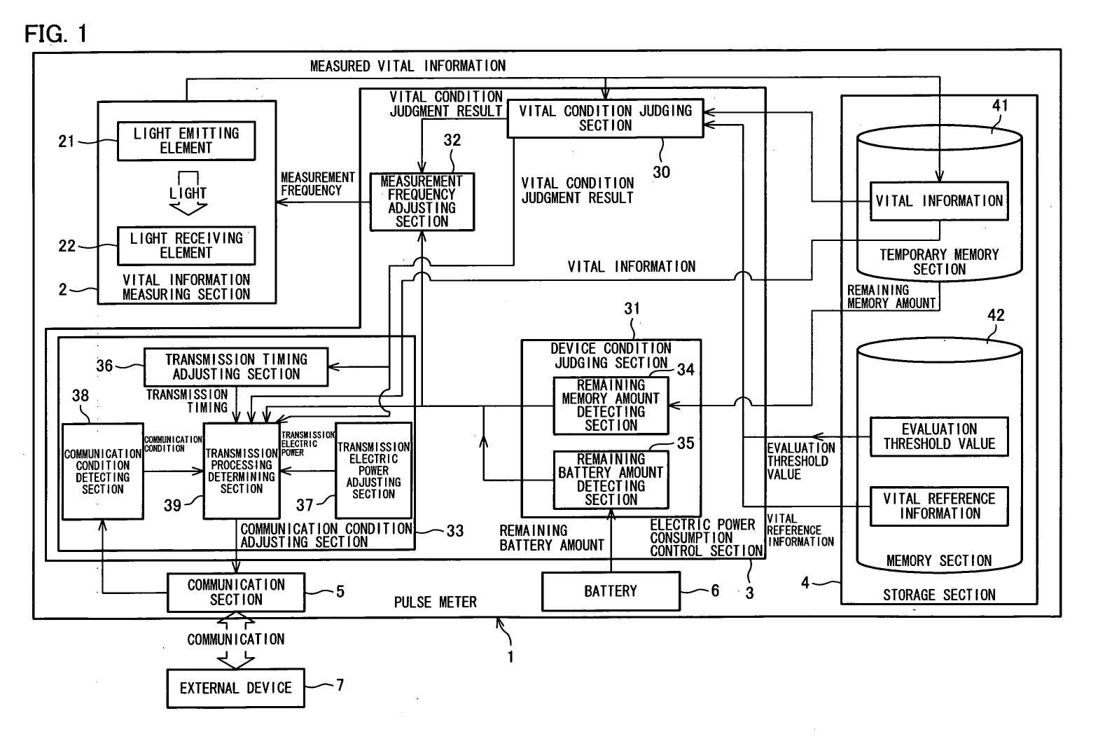

One embodiment of the present invention will be explained below with reference to FIG. 1 to FIG. 7. As one example of a vital information measuring device that is one embodiment of the present invention, the description herein describes a ring type pulse meter 1 (vital information measuring device) put on the vicinity of the root of a finger of a human organism so as to measure pulse rates (vital information) mainly from pulsation of an artery of the finger. However, the present invention is applicable not only to such a pulse meter but also to vital information measuring devices for measuring various vital information, such as a blood pressure meter and an oxygen saturation meter.

Further, the present invention is widely applicable to general measuring devices for measuring vital information and transmitting it via wireless or wired communication. Examples of such measuring devices are: a pulse meter, an electrocardiograph, a thermometer, a passometer, and a device for measuring a physical activity in accordance with acceleration, angular velocity, and the like. Further, the present invention is usable for a medical device used in a hospital or the like. Furthermore, the present invention is widely applicable to a health management system using the Internet.

(Configuration of Pulse Meter 1)

FIG. 1 is a function block diagram illustrating the configuration of the pulse meter 1, which is one example of the vital information measuring device that is one embodiment of the present invention. As shown in FIG. 1, the pulse meter 1 includes a vital information measuring section 2 (measuring means), an electric power consumption control section 3 (electric power consumption control means), a storage section 4, a communication section 5 (communication means), and a battery 6 (power source).

The vital information measuring section 2 serves as measuring means for measuring various vital information from the finger, which is a measurement target region, and includes a light emitting element 21 and a light receiving element 22 each for use in measuring a photoelectric pulse wave. The light emitting element 21 can be realized by, e.g., a light emitting diode (LED) or the like, and the light receiving element 22 can be realized by a photo diode (PD) or the like; however, the light emitting element 21 and the light receiving element 22 are not limited to these and may be realized by anything allowing for similar functions.

The vital information measuring section 2 is configured such that the light emitting element 21 irradiates detection light toward inside of a living organism and the light receiving element 22 receives the light thus irradiated and having passed through the living organism or having been reflected therein. Further, the vital information measuring section 2 converts the amount of light received by the light receiving element 22 (i.e., amount of a current flowing in the photo diode), into an analog voltage.

The analog voltage thus converted is further converted into digital data by an analog/digital converter (not shown) provided in the pulse meter 1, and calculation is carried out using the digital data. In this way, the vital information measuring section 2 functions as a photoelectric pulse sensor for detecting pulses, and outputs, as vital information, measurement data regarding the detected pulses.

As to a physical layout of the light emitting element 21 and the light receiving element 22, the light emitting element 21 and the light receiving element 22 may be provided face to face with each other such that the detection light irradiated from light emitting element 21 passes through the finger and the light receiving element 22 receives the light having passed therethrough. In this case, the vital information measuring section 2 is a transmissive type sensor. Alternatively, the light emitting element 21 and the light receiving element 22 are provided in the same plane and are directed in the same direction such that the light receiving element 22 receives reflected light of the light irradiated from the light emitting element 21 to the finger. In this case, the vital information measuring section 2 is a reflective type sensor. The light emitting element 21 and the light receiving element 22 are disposed inside a circular wearing portion (not shown), i.e., a ring portion of the ring type pulse meter 1 so as to be in close contact with the living organism when the vital information measuring section 2 is put on the finger.

The electric power consumption control section 3 controls electric power consumption of the pulse meter 1 in accordance with respective conditions of the living organism and the device. As shown in FIG. 1, the electric power consumption control section 3 includes a vital condition judging section 30 (vital condition judging means), a device condition judging section 31 (device condition judging means), a measurement frequency adjusting section 32 (measurement frequency adjusting means), and a communication condition adjusting section 33 (communication condition adjusting means).

The vital condition judging section 30 judges the vital condition of the user (living organism) in accordance with a comparison between (i) the vital information measured by the vital information measuring section 2 and (ii) information (vital evaluation information) stored in advance in a memory section 42 of the storage section 4 and used for evaluation of the vital condition of the living organism. An example of such vital evaluation information is an evaluation threshold value for evaluating the health condition of the user (living organism) (and vital reference information for improving accuracy of the evaluation). Here, the phrase “judging the vital condition of the living organism” means judging whether or not the vital information measured by the vital information measuring section 2 indicates a value representing a normal condition of the living organism (whether or not the living organism has abnormality). For example, the judgment as to the vital condition of the living organism is a judgment as to whether or not there is abnormality or a sign of abnormality in vital information representing the living organism's circulatory system condition, such as pulse rate, blood pressure, or oxygen saturation concentration in blood.

The device condition judging section 31 figures out the device condition of the pulse meter 1, for example, figures out whether a remaining memory amount in the pulse meter 1 is small or not and whether a remaining battery amount therein is small or not. Such a device condition judging section 31 includes a remaining memory amount detecting section 34 (remaining memory amount detecting means), and a remaining battery amount detecting section 35 (electric power remaining amount detecting means).

The device condition judging section 31 functions as device condition judging means for judging, in accordance with results of detections respectively carried out by the remaining memory amount detecting section 34 and the remaining battery amount detecting section 35, whether or not there is an alarming factor having an influence over a general operation of the pulse meter 1. The function of the device condition judging section 31 can be realized by software processing using an operation program for the electric power consumption control section 3, or may be realized by the software processing and a function of hardware such as an interrupt controller IC.

The remaining memory amount detecting section 34 manages a memory amount of a temporary memory section 41 (memory means) and a data amount of measurement data (vital information) stored in the memory, thereby detecting a remaining memory amount usable for storage in the memory. The function of the remaining memory amount detecting section 34 can be realized by the operation program for the electric power consumption control section 3.

When the device condition judging section 31 judges, in accordance with the detection result obtained by the remaining memory amount detecting section 34, that the remaining memory amount of the temporary memory section 41 is small, a transmission timing adjusting section 36 causes the communication section 5 to transmit data at an earlier timing or the measurement frequency adjusting section 32 causes the vital information measuring section 2 to carry out measurement less frequently. This makes it possible to avoid shortage of the memory amount in the temporary memory section 41 of the pulse meter 1.

The remaining battery amount detecting section 35 monitors an output voltage of the battery 6 so as to judge whether or not the output voltage is decreased to a predetermined voltage, thereby detecting a remaining battery amount. The remaining battery amount detecting section 35 can be realized by, e.g., a voltage detecting IC.

When the device condition judging section 31 judges, in accordance with the detection result obtained by the remaining battery amount detecting section 35, that, e.g., there is left a small amount of electric power that can be supplied from the battery 6, the below-described transmission timing adjusting section 36 delays a timing at which the communication section 5 transmits data, a transmission electric power adjusting section 37 causes transmission electric power for the communication section 5 to be smaller than the “normal electric power”, or the measurement frequency adjusting section 32 causes the vital information measuring section 2 to carry out measurement less frequently. This makes it possible to avoid shortage of an amount of electric power supply from the battery 6 to the pulse meter 1.

The measurement frequency adjusting section 32 appropriately adjusts how frequent the vital information measuring section 2 carries out measurement, in accordance with the judgment results of the vital condition judging section 30 and the device condition judging section 31, thus contributing to the electric power consumption control function of the electric power consumption control section 3.

The communication condition adjusting section 33 determines transmission electric power and a transmission processing method in accordance with (i) the judgment results of the vital condition judging section 30 and the device condition judging section 31, and (ii) a communication condition between the pulse meter 1 and an external device 7, and instructs the communication section 5 to carry out wireless communication therewith. The communication condition adjusting section 33 includes the transmission timing adjusting section 36 (transmission timing adjusting means), the transmission electric power adjusting section 37 (transmission electric power adjusting means), a communication condition detecting section 38, and a transmission processing determining section 39.

The transmission timing adjusting section 36 judges whether or not the predetermined data transmission timing set in advance has come, and adjusts the timing at which the communication section 5 carries out data communication, thus contributing to the electric power consumption control function of the electric power consumption control section 3.

Here, the phrase “adjust the timing at which the communication section 5 transmits the vital information” encompasses not only (i) a case of adjusting the timing in accordance with a condition of time information, such as a constant cycle or a fixed time, (ii) a case of adjusting the timing in accordance with a condition other than the time information, and (iii) a case of adjusting the timing in accordance with a combination of the above conditions.

The transmission electric power adjusting section 37 arbitrarily adjusts the strength, etc., of electric power used when the communication section 5 carries out data transmission, thus contributing to the electric power consumption control function of the electric power consumption control section 3. Generally, the strength of the transmission electric power can be set through a setting of a hardware register provided in an LSI (large-scale integration circuit) of an RF (Radio Frequency) module (not shown) constituting the communication section 5.

The communication condition detecting section 38 detects a communication condition between the communication section 5 and the external device 7, and judges whether or not data transmission has failed, whether or not the external device 7 is in a state in which the external device 7 can make communication, and the like.

The transmission processing determining section 39 comprehensively makes judgments in accordance with (i) the respective judgment results of the vital condition judging section 30 and the device condition judging section 31, (ii) the transmission timing determined by the transmission timing adjusting section 36, (iii) the transmission electric power determined by the transmission electric power adjusting section 37, and (iv) the communication condition, detected by the communication condition detecting section 38, of the pulse meter 1, and instructs the communication section 5 to transmit, to the external device 7, the vital information stored in the temporary memory section 41.

The communication section 5 functions as communication means for wirelessly transmitting the vital information, measured by the vital information measuring section 2, to the external device (external device 7) collecting vital information. The communication section 5 can be realized by using, e.g., a near field wireless communication method such as Bluetooth®, Zigbee®, the weak radio telemeter method, or the specified low power telemeter method.

The storage section 4 is made up of the temporary memory section 41 and a memory section 42, and exchanges various data with the vital information measuring section 2 and the electric power consumption control section 3. The present embodiment exemplifies that the temporary memory section 41 of the pulse meter 1 temporarily stores the vital information measured by the vital information measuring section 2, and the memory section 42 stores evaluation threshold values and vital reference information each described later.

The temporary memory section 41 functions as measurement data memory means for temporarily storing, as required, the measurement data, i.e., the vital information sent from the vital information measuring section 2, and can be realized by, e.g., a RAM (Random Access Memory), which allows for easy rewriting processing.

It is preferable to realize the memory section 42 by using, e.g., a rewritable nonvolatile memory such as an EEPROM (Electrically Erasable Programmable Read-only Memory) or a flash memory such that each of the evaluation threshold values can be appropriately updated to an adequate value and be stored therein. However, a RAM (Random Access Memory), which is a volatile memory, may be used for the memory section 42, and the electric power consumption control section 3 may write the evaluation threshold values in the RAM (Random Access Memory) every time the pulse meter 1 is powered on. Alternatively, a memory section storing the evaluation threshold values may be incorporated into the operation program for the electric power consumption control section 3 such that the evaluation threshold values are described as reference values in the program.

Further, the memory section 42 functions as vital reference information memory means for storing, as required, various vital reference information regarding the health of the user (living organism) of the vital information measuring device, which is one embodiment of the present invention. Note that the vital reference information may be stored as required, for the sake of improving accuracy in the judgment carried out by the vital condition judging section 30. Therefore, the vital reference information is not necessarily required.

The communication section 5 is a section having a function as communication means for communicating with the external device.

It is assumed in the present embodiment that the communication section 5 and the external device 7 communicate with each other wirelessly; however, the communication therebetween is not limited to wireless communication, and may be wired communication.

The battery 6 is a power source for supplying electric power to the pulse meter 1, and is required to be small but have a large capacity as much as possible in the ring type pulse meter 1 of the present embodiment, which is a small vital information measuring device that carries out measurement on regular basis. For this reason, it is preferable to realize the battery 6 by, e.g., a button type primary battery. However, the battery 6 is not limited to such a button type primary battery, and may be any battery that exhibits the same function, such as a secondary battery.

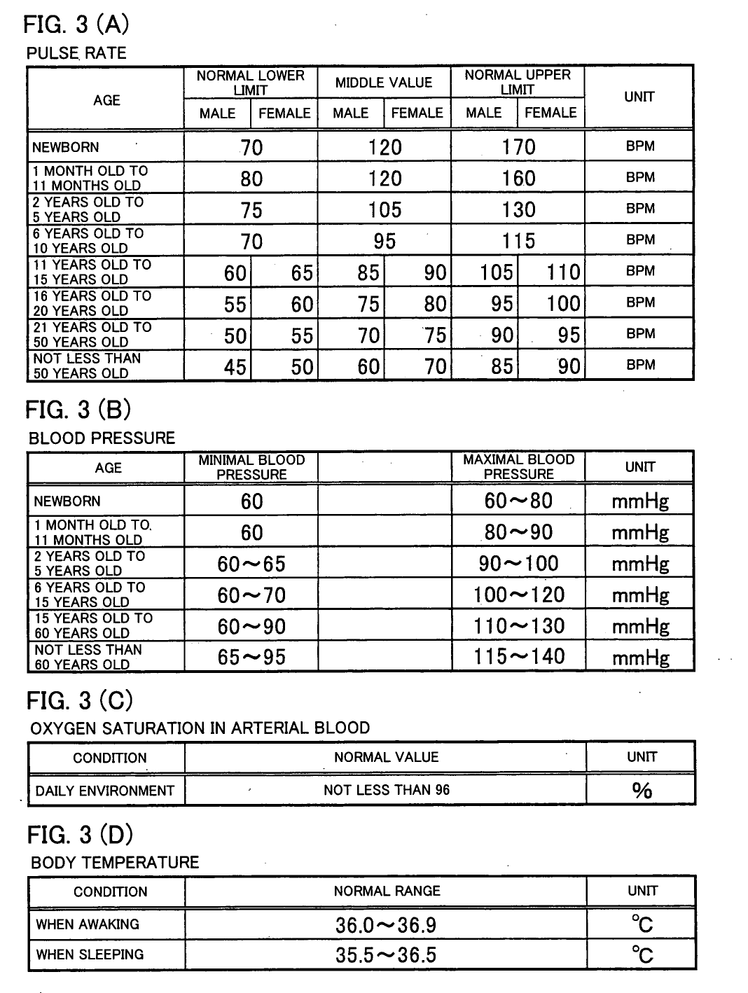

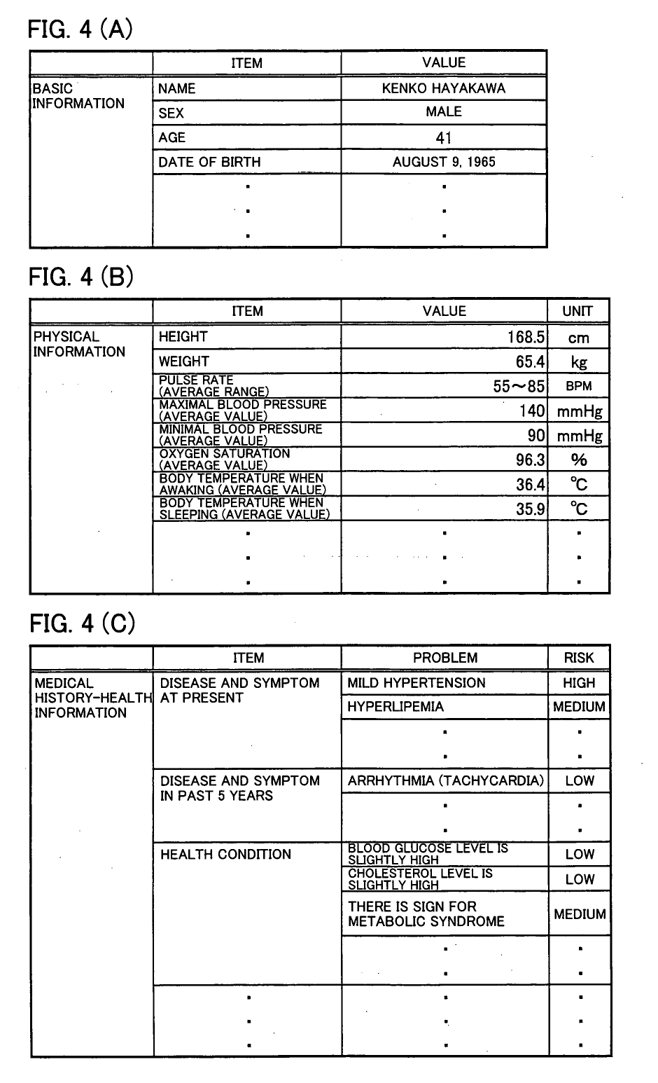

The following explains specific examples of the evaluation threshold values and the vital reference information, with reference to FIG. 3(A) to FIG. 3(D) and FIG. 4(A) to FIG. 4(C). Explained first are specific examples of the evaluation threshold values. The table of FIG. 3(A) shows examples of evaluation threshold values for pulse rate. Specifically, the table defines, based on ages and sexes, normal lower limit values (abbreviated as “NORMAL LOWER LIMIT” in FIG. 3(A)) for pulse rate, middle values therefor, and normal upper limit values (abbreviated as “NORMAL UPPER LIMIT” in FIG. 3 (A)) therefor. For example, consider a case where the user's age is “17” and the user's sex is “male”. In this case, with reference to the column corresponding to “16 YEARS OLD TO 20 YEARS OLD”, it is found that the normal lower limit value of the normal pulse rate for the user is “55 bpm (beat per minute)”, the middle value thereof is “75 bpm”, and the normal upper limit value thereof is “95 bpm”.

As reference examples of evaluation threshold values other than those for pulse rate, FIG. 3(B) shows examples of evaluation threshold values for blood pressure, FIG. 3(C) shows examples of evaluation threshold values for oxygen saturation in arterial blood, and FIG. 3(D) shows examples of evaluation threshold values for body temperature. These information are arbitrarily acquired from, e.g., the external device 7 via the communication section 5 and are stored in the memory section 42.

Now, see FIG. 3(B) for the examples of the evaluation threshold values for blood pressure. For example, consider a case where the user's age is “17” and the user's sex is “male”. In this case, with reference to the column corresponding to “15 YEARS OLD TO 60 YEARS OLD”, it is found that the minimal normal blood pressure for the user is “60 mmHg to 90 mmHg”, and the maximal normal blood pressure therefor is “110 mmHg to 130 mmHg”. Meanwhile, see FIG. 3(C) for the examples of the evaluation threshold values for oxygen saturation in arterial blood. For example, in cases where oxygen saturation in arterial blood is “98%” under daily environment, the oxygen saturation is judged to be normal. Meanwhile, see FIG. 3(D) for the examples of the evaluation threshold values for body temperature. In cases where the user's body temperature is “36.6° C.” while the user is awake, the body temperature is judged to be normal.

Next, see FIG. 4(A) to FIG. 4(C) for the specific examples of the vital reference information. FIG. 4(A) shows examples of “basic information” regarding a living organism, such as name, sex, age, and date of birth. The basic information does not have a direct relation with the living organism's health condition, but is information representing attributes intrinsic to the living organism. Such basic information is stored in the memory section 42 shown in FIG. 1. The basic information is acquired from, e.g., the external device 7 via the communication section 5 and is stored in the memory section 42.

FIG. 4(B) shows examples of “physical information” regarding the living organism, such as the living organism's height, weight, pulse rate (average range), maximal blood pressure (average value), minimal blood pressure (average value), oxygen saturation (average value), body temperature when awaking (average value), and body temperature when sleeping (average value). As such, the physical information is information that is based on information measured from the physical body of the living organism and that indicates the living organism's recent physical condition. Such physical information is stored in the memory section 42.

In the pulse meter 1, the pulse rate (average range) among these information may be found through calculation using measurement data obtained hitherto (the living organism's pulse rates in past and at present). For example, as a result of the calculation, the average value of the pulse rates below the middle value between the evaluation threshold values in FIG. 3(A) is set to be the lower limit, and the average value of the pulse rates above the middle value therebetween is set to be the upper limit. A range from the lower limit to the upper limit of the pulse rates is the average range of the pulse rates. In this way, it is possible to set the average range of the pulse rates only for the living organism in accordance with the history of measurement.

Note that the information such as the maximal blood pressure (average value), the minimal blood pressure (average value), the oxygen saturation (average value), the body temperature when awaking (average value), and the body temperature when sleeping (average value) are obtained by other vital information measuring devices than the pulse meter 1 and health-related devices, are acquired from the external device 7 via the communication section 5 appropriately, and are stored in the memory section 42.

Further, the pulse rate (average range) may be acquired from the external device 7 via the communication section 5. For example, by acquiring a pulse rate (average range) set by a medical institution etc., to which the living organism goes and adequate for judging the living organism's health condition, it is possible for the vital condition judging section 30 to make an adequate judgment in terms of medical sense.

The table of FIG. 4(C) shows examples of “medical history-health information” regarding the living organism. Specifically, the medical history-health information is information regarding the living organism's health management, such as (i) medical history such as diseases and symptoms that the living organism is suffering at present and has suffered in past and (ii) supplementary information regarding the living organism's health condition (e.g., knowledge from the medical institute, etc., to which the living organism goes). Such medical history-health information is stored in the memory section 42.

As described above, the indexes regarding circulatory system such as blood pressure and pulsation, or physical indexes such as the number of steps in walk, acceleration, and body temperature may be used for indication of the vital condition. Further, the reference information are managed based on the categories of the attributes such as the user's age and sex, and the vital condition judging section 30 makes a judgment in accordance with the reference information. This makes it possible to make an adequate judgment based on the categories of the attributes.

Apart from these examples, there may be provided various vital reference information regarding the physical body and health of the user (living organism) and regarding the physical body and health of a living organism other than the user, such as (i) the user's parents, grandparents, children, and relatives who are quite similar to the user physically and (ii) a living organism having physical characteristics and constitution similar to the user. This makes it possible for the vital condition judging section 30 to make a judgment finely.

Note that the information (vital evaluation information) for evaluating the living organism is not limited to (i) the “evaluation threshold values”, which are threshold values for evaluating the vital condition of the living organism, and (ii) the combination of the “evaluation threshold values” and the “vital reference information”, which is referred for more accurate judgment. The vital evaluation information may be any information allowing for evaluation of the vital condition of the living organism.

(Operation of Pulse Meter 1)

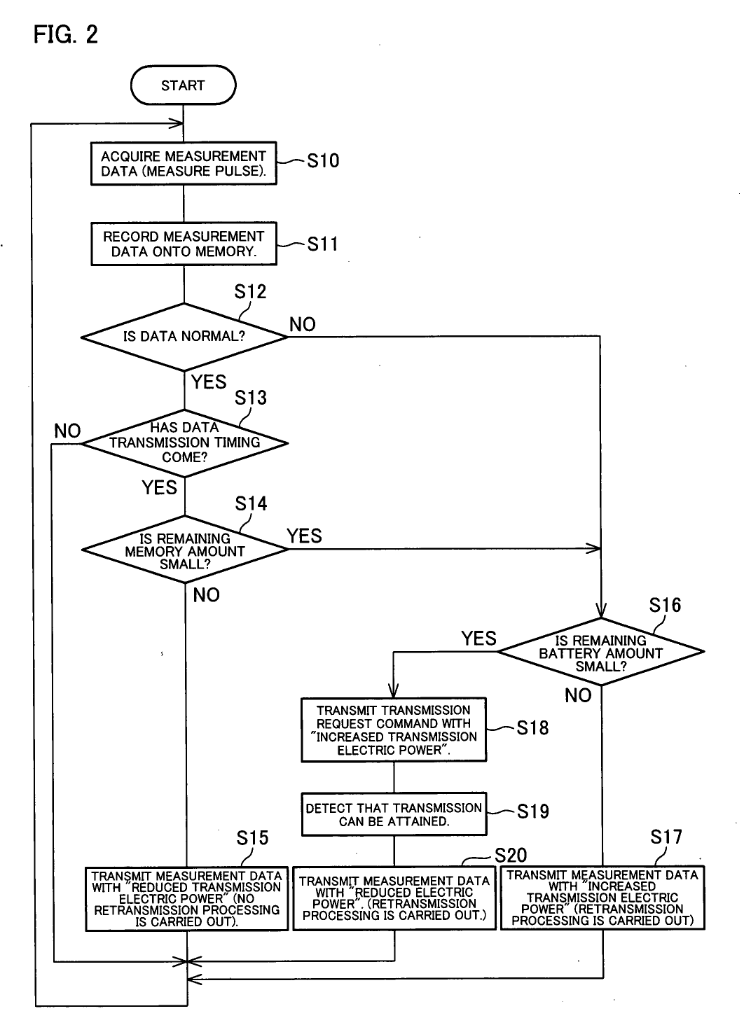

Explained next is how the pulse meter 1 of the present embodiment operates, with reference to FIG. 1 and FIG. 2. FIG. 2 is a flowchart illustrating the operations of the pulse meter 1 of the present embodiment.

See FIG. 2. In the first step, i.e., Step 10 (hereinafter, abbreviated as “S10”), the vital information measuring section 2 measures the living organism's pulse rate (vital information). The pulse meter 1 is configured such that, e.g., the measurement frequency adjusting section 32 shown in FIG. 1 controls how frequent and how many times the living organism's pulse rate are going to be measured. The measurement may be carried out at arbitrary timings in accordance with a purpose of the measurement and the user's (living organism's) settings. For example, the measurement is determined to be carried out every 1 minute, 10 minutes, or 1 hour. Alternatively, the measurement can be appropriately carried out in response to a request received from the external device 7 via the communication section 5.

The following may carried out for reduction of consumption of electric power required for the measurement (electric power required for light emission of the light emitting element 21; electric power required for respective operations of the electric power consumption control section 3, the vital information measuring section 2, and so on). That is, in cases where the values of the pulse rates (vital information) stored and accumulated in the temporary memory section 41 are sufficiently away from the evaluation threshold values or where the values of the pulse rates are in the vicinity of the middle value of the daily pulse rates (average range), the vital condition judging section 30 judges that the condition of the user (living organism) is normal and stable. In accordance with the result of this judgment, the measurement frequency adjusting section 32 determines that the measurement is to be carried out less frequently (e.g., the measurement is to be carried out every 30 minutes although the measurement is set to be carried out in every 10 minutes normally).

With this, wasting of the battery 6 due to frequent measurement operations is reduced. As a result, the measurement frequency adjusting section 32 contributes to reduction of electric power consumption of the pulse meter 1. Note that when the measurement frequency adjusting section 32 judges, in accordance with a judgment result of the device condition judging section 31, that the device condition of the pulse meter 1 is abnormal, the measurement frequency adjusting section 32 may determine that the measurement is carried out less frequently.

Namely, in accordance with the respective judgment results, etc., of the vital condition judging section 30 and the device condition judging section 31, the measurement frequency adjusting section 32 may adjust how frequent the measurement is carried out.

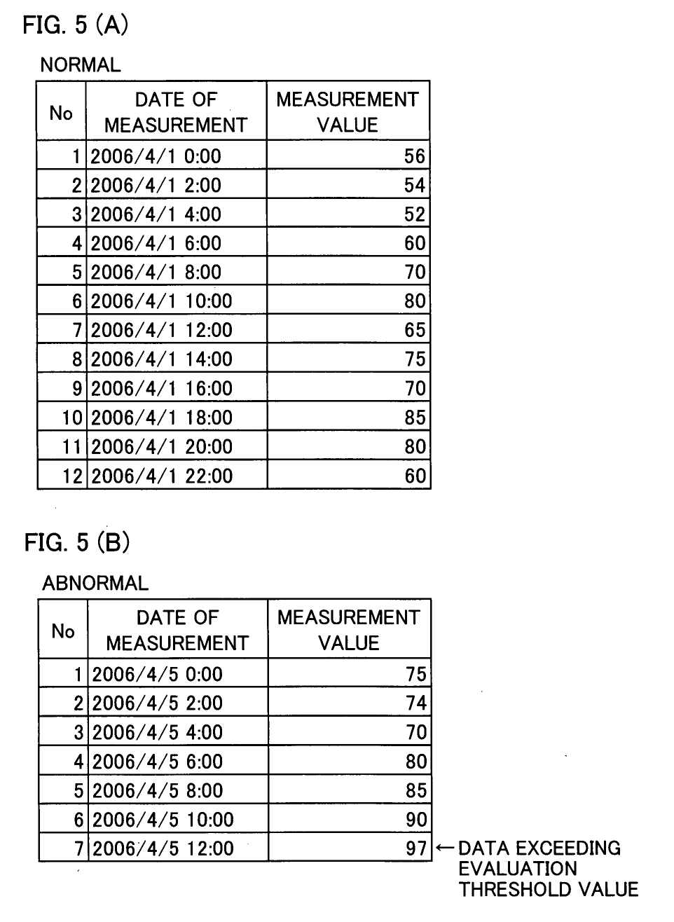

Next, in S11 shown in FIG. 2, the measurement data representing the pulse rate (vital information) measured in S10 is recorded onto the temporary memory section 41 shown in FIG. 1. Measurement data, which respectively indicate dates of measurement and measurement values, are stored and accumulated in the temporary memory section 41 in the order of, e.g., time as shown in FIG. 5(A).

In the example shown in FIG. 5(A), the measurement data measured every fixed time are stored and accumulated in the order from the measurement data oldest in the date of measurement; however, the present invention is not limited to this. The measurement data may be stored and accumulated in an appropriate manner in accordance with a purpose of the measurement and the user's (living organism's) settings. For example, a predetermined amount of measurement data from the newest one to an older one may be stored. Alternatively, only measurement data having changed in measurement values may be stored and accumulated.

By thus storing and accumulating, in the temporary memory section 41, only the measurement data having changed in measurement values, the amount of data stored and accumulated therein is reduced as much as possible, with the result that it takes longer time that the amount of data stored in the temporary memory section 41 reaches a predetermined data amount (e.g., data amount corresponding to 100 samples). This delays a timing of transmission operation, which is carried out when the amount of data stored therein reaches the predetermined amount. Accordingly, wasting of the battery 6 due to frequent transmission operations is reduced.

Next, in S12 shown in FIG. 2, the vital condition judging section 30 shown in FIG. 1 compares (i) the measurement data indicating the pulse rate (vital information) shown in FIG. 2 and measured in S10 with (ii) the evaluation threshold values stored in the memory section 42 shown in FIG. 1, so as to judge whether or not the pulse rate is a normal value (whether or not there is abnormality in the living organism).

For the purpose of making an accurate judgment, the vital reference information stored in the memory section 42 is used in the present embodiment. Here, the following concretely explains the judgment with reference to the exemplary evaluation threshold values shown in FIG. 3(A) and the exemplary vital reference information shown in FIG. 4(A) and FIG. 4(B). In FIG. 3(A), the normal lower limit values, the middle values, and the normal upper limit values for pulse rate are defined based on ages and sexes.

In cases where there are the evaluation threshold values thus finely classified based on ages and sexes, it is possible to selectively use a more accurate evaluation threshold value with reference to the living organism's attributes “sex” and “age” of the basic information exemplified in FIG. 4(A). In other words, in the example of FIG. 4(A), the living organism's sex is “male” and age is “41”. With reference to the column “21 YEARS OLD TO 50 YEARS OLD” shown in FIG. 3(A), it is found that the normal lower limit value of the evaluation threshold values for the living organism is “50 bpm”, the middle value thereof is “70 bpm”, and the normal upper limit value thereof is “90 bpm”. In cases where the measured pulse rate is “88 bpm”, the measured pulse rate falls within the normal range of the evaluation threshold values, so that the vital condition judging section 30 judges “Normal” (YES).

Further, the “pulse rate (average range)” of the “physical information” exemplified in FIG. 4(B) may be used.

Assume that the user's recent (daily) pulse rate (average range) falls within a range from “55 bpm to 85 bpm” as shown in FIG. 4(B). Even though the measured pulse rate is “88 bpm” falling within the normal range of the evaluation threshold values shown in FIG. 3(A), the pulse rate of “88 bpm” is higher than the user's daily average range. In this case, the vital condition judging section 30 recognizes that there is a sign of abnormality and judges “Abnormal” (NO), thereby drawing the living organism's attention to the health condition thereof.

Further, by using the “medical history-health information” of FIG. 4(C) for the vital reference information, it is possible to make a judgment in consideration of the living organism's medical history and health condition. For example, assume that the measured pulse rate is “80 bpm”. In this case, the vital condition judging section 30 judges “Normal” (YES) even when the aforesaid “pulse rate (average range)” is used.

However, in cases where the risk of “mild hypertension” due to health condition of circulatory system is indicated to be “high” in “DISEASE AND SYMPTOM AT PRESENT” shown in FIG. 4(C), the vital condition judging section 30 judges “Abnormal” (NO) when the measurement value is around the upper limit of the daily average range, thereby drawing the living organism's attention to the health condition thereof.

The vital condition judging section 30 judges “Abnormal” (NO) when the measurement value is around the upper limit of the daily average range, not only in the case where the risk of “mild hypertension” is indicated to be “high” but also in cases where “DISEASE AND SYMPTOM IN PAST 5 YEARS” indicates “arrhythmia (tachycardia)” or where “HEALTH CONDITION” indicates that “there is a sign of metabolic syndrome”. In this way, it is possible to draw the living organism's attention to the health condition thereof.

As such, the judgment in S12 shown in FIG. 2 is carried out by using the vital reference information, with the result that the judgment is carried out appropriately in accordance with the living organism's attributes, recent physical condition, medical history, health condition, risk in the health condition, and the like.

Next, consider a case where the living organism has been extremely healthy from past to present and there is therefore no problem even if some physical loads are imposed on the living organism, unlike the above case where the vital condition judging section 30 judges “Abnormal” (NO) in accordance with the living organism's medical history even when the measured pulse rate is normal in view of the evaluation threshold values.

In this case, even when the measured pulse rate exceeds the evaluation threshold value and the user's daily pulse rate (average range), the judgment value is raised (e.g., up to the upper limit value+10 bpm) such that the vital condition judging section 30 judges “Normal” (YES).

This makes it possible to restrain, as required, communication for making a notification supposed to be urgent under normal circumstances, with the result that electric power consumption required for the communication is reduced and notifications are not made excessively and more than necessary. Accordingly, it is possible to provide such a vital information measuring device, which is configured to arbitrarily read out the vital reference information from the external device 7 and operates based on the living organism's characteristics.

Next, in cases where the judgment result in S12 shown in FIG. 2 is “YES” (i.e., “Normal”), the sequence goes to S13. Specifically, the vital condition judging section 30 shown in FIG. 1 notifies the judgment result (“Normal”) regarding the vital information to the transmission timing adjusting section 36 of the communication condition adjusting section 33. In the present embodiment, the judgment result is notified to the transmission timing adjusting section 36 as such; however, the receiving end of the notification is not limited to the transmission timing adjusting section 36, and may be determined appropriately as required.

For example, the vital condition judging section 30 may notify the judgment result to the transmission processing determining section 39, and the transmission processing determining section 39 thus notified instructs the transmission timing adjusting section 36 to judge whether or not a data transmission timing has come.

Note that the vital condition judging section 30 may notify, to the transmission timing adjusting section 36, not only the judgment result “Normal”, but also information indicating a judged level regarding how normal the vital information is. With this, the judgment process can be more adequately carried out by the transmission timing adjusting section 36 in S13 described below. For example, in cases where the value of the measured pulse rate falls within the normal range but is close to the maximal value of the average range of the daily pulse rate (“physical information” of the aforesaid vital reference information), the vital condition judging section 30 notifies thereto a judged level “Small Margin” for the purpose of drawing the user's (living organism's) attention at an early stage.

Here, the judged level “Small Margin” indicates that the living organism is not in the abnormal condition but is in a condition close to the abnormal condition. Whether or not the judged level is “Small Margin” may be judged, e.g., as follows. That is, in the case of pulse rate, pulse rate threshold values different from the evaluation threshold values are set at values close to the values of the abnormal pulse rates respectively, and a judgment is made as to whether or not the measured pulse rate goes beyond or falls below the pulse rate threshold values thus set.

In contrast, when the value of each data representing a pulse rate (vital information) and stored and accumulated in the temporary memory section 41 shown in FIG. 1 is sufficiently away from the evaluation threshold values or is around the middle value of the daily pulse rate (average range), the vital condition judging section 30 judges that the user's (living organism's) condition is normal and stable, and therefore notifies thereto a judged level “Large Margin”.

Here, the judged level “Large Margin” indicates that the condition of the living organism is sufficiently away from the abnormal condition. Whether or not the judged level is “Large Margin” may be judged, e.g., as follows. That is, in the case of pulse rate, an upper limit value and a lower limit value different from the evaluation threshold values respectively are set in the vicinity of the middle value of the daily pulse rate (average range), and judgment is made as to whether or not the pulse rate always falls within a range between the set upper limit value and lower limit value during a predetermined period of time.

The above expressions regarding the judged level are mere examples. Apart from the examples, the value of a difference between the measurement value and each of the evaluation threshold values (e.g., “lower limit value+5”, “upper limit value−10”, or the like) may be used therefor, for example. Hence, information indicating an appropriate judged level may be notified in accordance with the judgment result of the transmission timing adjusting section 36.

In the meanwhile, the judgment result in S12 is “NO” (i.e., “Abnormal”), the sequence goes to S16 in FIG. 2. Specifically, the vital condition judging section 30 shown in FIG. 1 notifies the judgment result (“Abnormal”) to the transmission processing determining section 39 and the transmission processing determining section 39 instructs the device condition judging section 31 to transmit the judgment result regarding the remaining battery amount.

In S13 shown in FIG. 2, the transmission timing adjusting section 36 shown in FIG. 1 judges whether or not the predetermined data communication timing has come. Specifically, the “predetermined data communication timing” refers to a timing indicated by time information, such as a timing coming in a certain cycle (e.g., every 15 minutes) or a certain time (e.g., 0:00 am and 0:00 pm). Whether or not the predetermined data communication timing has come is judged by carrying out monitoring in accordance with the time information with the use of a timer (not shown) or a clock (not shown) provided in the pulse meter 1.

Now, consider a case where the measurement is carried out irregularly, for example. In this case, whether or not the predetermined data communication timing has come is judged by using the following condition other than the time information: (i) whether or not the amount of measurement data stored in the temporary memory section 41 reaches the predetermined data amount (e.g., data amount corresponding to 100 samples); (ii) whether or not the remaining memory amount detecting section 34 detects that a remaining memory amount in the temporary memory section 41 is not more than a predetermined memory amount; or the like.

Further, as the condition other than the time information, the judged level notified from the vital condition judging section 30 may be used. For example, in cases where the pulse rate measured in S10 shown in FIG. 2 falls within the normal range but is close to the maximal value of the average range of the daily pulse rate (the “physical information” of the aforesaid vital reference information), the judged level “Small Margin” is notified to the transmission timing adjusting section 36. In response to the notification, the transmission timing adjusting section 36 judges that the data communication timing has come, for the purpose of drawing the living organism's attention at an early stage.

Here, further explanation regarding the predetermined data transmission timing is made. In cases where the judgment result for the measurement data in S12 shown in FIG. 2 is “Normal” (there is no abnormality in the living organism), the data does not need to be transmitted immediately. Hence, for reducing wasting of the battery due to frequent wireless communication, it is preferable that measurement data be accumulated as much as possible and the data communication timing be adjusted at an appropriate timing by the transmission timing adjusting section 36 such that the measurement data are transmitted all together at the predetermined data communication timing.

As such, for reducing the electric power consumption in communication, the judged level, notified from the vital condition judging section 30 and indicating “Large Margin” when the user's (living organism's) condition is judged to be normal and stable, is used while the timing coming in the “certain cycle” or “certain time” is employed as the predetermined data communication timing. On this account, even when the judged level indicates “Large Margin”, the transmission timing adjusting section 36 judges “NO”, i.e., judges that the data communication timing has not come yet, even though the timing coming in the “certain cycle” or “certain time” has actually come. Accordingly, the measurement data is accumulated as long as there is left a sufficient memory amount in the temporary memory section 41, with the result that communication is less frequently carried out. Examples of the case where the user's (living organism's) condition is judged to be normal and stable include: (i) a case where the value of the data indicating the pulse rate (vital information) and stored in the temporary memory section 41 shown in FIG. 1 is sufficiently away from the evaluation threshold value; (ii) a case where the value thereof is in the vicinity of the middle value of the daily pulse rate (average range); and the like. In accordance with the plurality of conditions combined in this way, the transmission timing adjusting section 36 may judge whether or not the predetermined data transmission timing has come.

In cases where the judgment result obtained in S13 shown in FIG. 2 is “YES”, i.e., where the predetermined data communication timing has come, the sequence goes to S14. Specifically, the transmission timing adjusting section 36 notifies the transmission processing determining section 39 that the data communication timing has come. On the other hand, in cases where the judgment result therein is “NO”, i.e., where the data communication timing has not come, the sequence goes back to S10, the initial state (the step of measuring a pulse rate).

In S14, when the transmission processing determining section 39 shown in FIG. 1 is notified by the transmission timing adjusting section 36 that the transmission timing has come, the transmission processing determining section 39 instructs the device condition judging section 31 to transmit thereto a result of judging the remaining memory amount. Then, the device condition judging section 31 thus instructed judges whether or not the remaining memory amount of the temporary memory section 41 detected by the remaining memory amount detecting section 34 is not more than the predetermined memory amount, and notifies the result thereof to the transmission processing determining section 39.

In cases where the judgment result obtained in S14 shown in FIG. 2 is “NO”, i.e., where the remaining memory amount is not less than the predetermined memory amount, the sequence goes to S15. Specifically, the transmission processing determining section 39 shown in FIG. 1 instructs the transmission electric power adjusting section 37 to determine transmission electric power.

On the other hand, in cases where the judgment result in S14, shown in FIG. 2 is “YES”, i.e., where the remaining memory amount is not more than the predetermined memory amount, the sequence goes to S16. Specifically, the transmission processing determining section 39 shown in FIG. 1 instructs the device condition judging section 31 to transmit thereto a result of judging the remaining battery amount.

S15 shown in FIG. 2 is not a step performed in the case of emergency, i.e., is a normal transmission processing step. In S15, the transmission processing determining section 39 causes the communication section 5 to transmit, to the external device 7, the measurement data temporarily stored in the temporary memory section 41 in S11, accumulated until the predetermined transmission timing comes, and indicating the pulse rates (vital information). In accordance with the electric power determined by the transmission electric power adjusting section 37, the transmission processing determining section 39 instructs the communication section 5 to transmit the measurement data with the transmission electric power reduced.

The description “with the transmission electric power reduced” herein indicates that the electric power is reduced such that the electric power is relatively “weaker” than transmission electric power used in S17 and S18 shown in FIG. 2 as described later. Namely, the electric power thus reduced is either (i) the minimal electric power by which communication between the pulse meter 1 (vital information measuring device) and the external device 7 is attained in normal use with a physical distance therebetween, or (ii) electric power close to the minimal electric power. In cases where the pulse meter 1 of the present embodiment is put on the finger of the living organism (human organism) and the measurement data is to be transmitted to a mobile phone usually carried by the living organism and serving as the external device 7 (data collecting device), the electric power is adjusted to be electric power allowing for communication within a distance of, e.g., approximately 1 m to approximately 2 m with nothing interposed therebetween.

Apart from the above case, for example, the “electric power thus reduced” may be (i) the minimal one of “normal electric power”, each of which is defined to be transmission electric power falling with in a range from the maximal transmission electric power to the minimal electric power each allowing for communication between the pulse meter 1 and the external device 7 separated from each other by a physical distance assumed in normal use (distance assumed to be maximal, distance assumed to be minimal); or (ii) transmission electric power close to the minimal normal electric power.

Note that the transmission electric power “close to the minimal normal electric power” refers to transmission electric power smaller than the average value of the maximal transmission electric power and the minimal transmission electric power.

That is, the transmission electric power may be set at minimally required transmission electric power or transmission electric power close to the minimally required transmission electric power in consideration of (i) actual use conditions of the vital information measuring device and the external device (physical distance therebetween, body-wearing conditions, installation conditions, placement conditions, and the like in normal use); (ii) antenna properties; (iii) specification (reliability in communication) required for communication between the vital information measuring device and the external device; and the like.

In cases where the transmission of the measurement data has failed (e.g., where the pulse meter 1 cannot receive a response sent from the external device 7 and indicating that reception has been made), arbitrary processing such as retransmission or non-retransmission may be carried out in accordance with (i) a communication specification between the pulse meter 1 and the external device 7 (e.g., specification of a communication protocol of the high layer, i.e., the application layer), and (ii) the specification (e.g., retransmission processing, etc., with the Bluetooth® protocol stack) of a communication method implemented in the communication section 5. A specific example of the communication method is Bluetooth®.

However, the retransmission processing in communication consumes electric power, thereby wasting the battery additionally. Hence, in cases where the retransmission does not need to be carried out urgently, it is generally advantageous, in terms of low electric power consumption, that the measurement data measured currently is not retransmitted at the current predetermined transmission timing but will be transmitted at the next transmission timing together with the measurement data to be measured next.

For this reason, in the present embodiment, in cases where the transmission of the measurement data has failed, the communication condition detecting section 38 detects the failure of the transmission, and notifies it to the transmission processing determining section 39. The transmission processing determining section 39 thus notified retains the measurement data in the temporary memory section 41 and the measurement data thus retained is to be transmitted in the next transmission timing in accordance with determination of the transmission timing adjusting section 36.

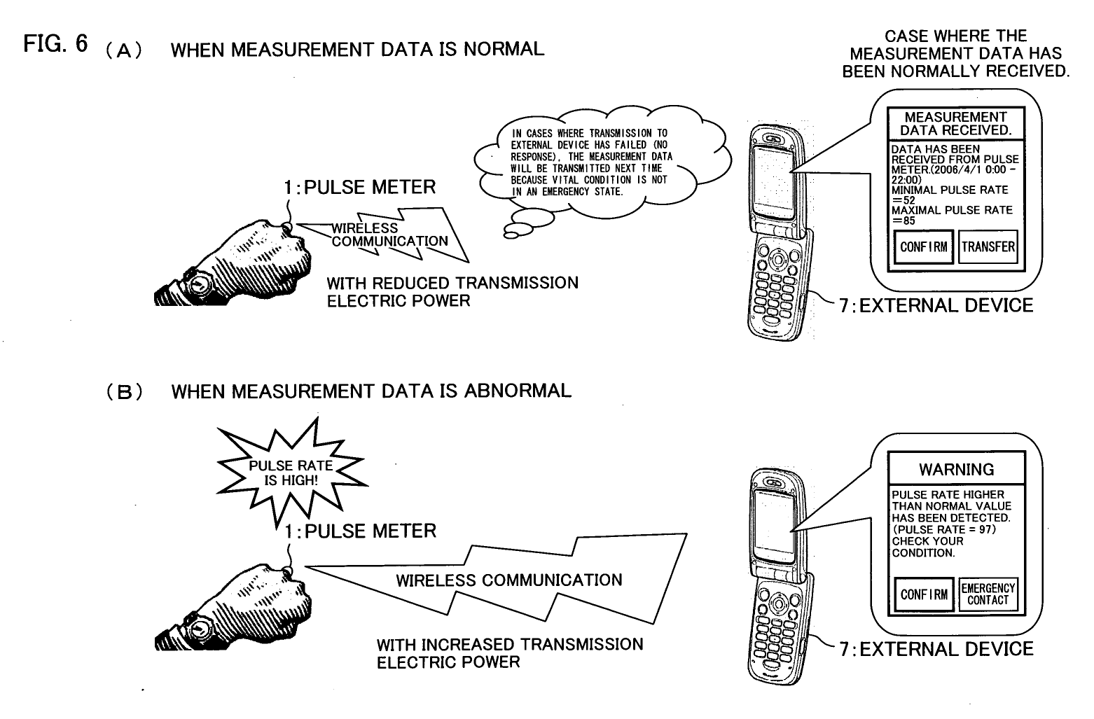

FIG. 6(A) illustrates an example of presenting, to the living organism (user), the measurement data on a screen of the external device 7 that is shown in FIG. 1 and has normally received the measurement data indicating “Normal” as shown in FIG. 5(A) and transmitted from the pulse meter 1 with the reduced transmission electric power.

In this example of presentation, when the living organism selects “CONFIRM” displayed on the lower left portion of the screen, the display of the measurement data is terminated and the measurement data is stored in the external device 7. On the other hand, when the living organism selects “TRANSFER” displayed on the lower right portion of the screen, the external device 7 transfers the measurement data to a predetermined destination such as a host server or the like.

After the processing in S15 shown in FIG. 2, the sequence goes back to S10. Specifically, the sequence goes back to the initial state as with the above case. In S16, the device condition judging section 31 shown in FIG. 1 judges whether or not the battery amount left in the battery 6 and detected by the remaining battery amount detecting section 35 is not more than the predetermined amount.

In cases where the result of the judgment in S16 shown in FIG. 2 is “NO”, i.e., where the remaining battery amount is not less than the predetermined amount, the sequence goes to S17. Specifically, the judgment result obtained by the device condition judging section 31 shown in FIG. 1 and indicating that there is left a sufficient battery amount is notified to the transmission processing determining section 39. On the other hand, in cases where the result is “YES”, i.e., where the remaining battery amount is not more than the predetermined amount, the sequence goes to S18. Specifically, the judgment result obtained by the device condition judging section 31 shown in FIG. 1 and indicating that there is not left a sufficient battery amount is notified to the transmission processing determining section 39.

S17 shown in FIG. 2 is a transmission processing step performed in cases where the remaining battery amount is not less than the predetermined amount and in the case of emergency. In S17, the transmission processing determining section 39 causes the communication section 5 to transmit, to external device 7, the measurement data, which have been temporarily stored and accumulated in S11 in the temporary memory section 41 shown in FIG. 1 and respectively indicate the pulse rates (vital information).

On this occasion, the transmission processing determining section 39 instructs the transmission electric power adjusting section 37 to determine transmission electric power. Then, in accordance with the determination of the transmission electric power adjusting section 37, the transmission processing determining section 39 causes the vital information to be transmitted with the transmission electric power increased.

The description “with the transmission electric power increased” herein indicates that the electric power is increased to be relatively “stronger” than the transmission electric power used in S15 described above and S18 described below. Namely, the transmission electric power is increased to be transmission electric power used in cases where the physical distance between the pulse meter 1 (vital information measuring device) and the external device 7 is (i) the maximal distance allowing for communication therebetween (e.g., in the case of Bluetooth® class 3 device, a distance of 10 m with nothing interposed therebetween) or (ii) a distance close to the maximal distance.

In cases where the pulse meter 1 of the present embodiment is put on the finger of the living organism (human organism) and the measurement data is to be transmitted to the mobile phone serving as the external device 7 (data collecting device) and positioned near the living organism's body, the transmission electric power is adjusted to, e.g., transmission electric power securely allowing for communication in a room of a general house, i.e., communication in a distance of 5 m to 10 m with nothing interposed therebetween.

Apart from the above case, for example, the “electric power thus increased” may be (i) the maximal one of the “normal electric power”, each of which is defined to be transmission electric power falling with in a range from the maximal transmission electric power to the minimal electric power each allowing for communication between the pulse meter 1 and the external device 7 separated from each other by a physical distance assumed in normal use (distance assumed to be maximal, distance assumed to be minimal); or (ii) transmission electric power close to the maximal normal electric power.

Note that the transmission electric power “close to the maximal normal electric power” refers to transmission electric power larger than the average value of the maximal transmission electric power and the minimal transmission electric power. That is, the transmission electric power may be set at minimally required strong transmission electric power for the sake of restraining electric power consumption as much as possible, in consideration of (i) the actual use conditions of the vital information measuring device and the external device (physical distance therebetween, body-wearing conditions, installation conditions, placement conditions, and the like, in normal use); (ii) antenna properties; (iii) specification (reliability in communication) required for communication between the vital information measuring device and the external device; and the like.

In cases where the transmission of the measurement data has failed (e.g., where the pulse meter 1 cannot receive a response sent from the external device 7 and indicating that reception has been made), arbitrary processing such as retransmission or non-retransmission may be carried out in accordance with (i) the communication specification between the pulse meter 1 and the external device 7 (e.g., specification of the communication protocol of the high layer, i.e., the application layer), and (ii) the specification (e.g., retransmission processing in the Bluetooth® protocol stack) of a communication method implemented in the communication section 5. A specific example of the communication method is Bluetooth®.

However, the transmission electric power is increased in S17 shown in FIG. 2, for the sake of carrying out transmission as secure as possible and as prompt as possible. For this reason, in the present embodiment, in cases where the transmission of the measurement data has failed, the communication condition detecting section 38 shown in FIG. 1 detects the failure and notifies it to the transmission processing determining section 39.

The transmission processing determining section 39 thus notified causes the communication section 5 to retransmit the measurement data to the external device 7 with the increased transmission electric power maintained, in accordance with determination of the transmission electric power adjusting section 37. In cases where the retransmission has failed even though the predetermined retransmission processing is carried out, the living organism is notified through, e.g., display or a sound from a notifying section (not shown), which may be provided in the pulse meter 1.

FIG. 6(B) illustrates an example of presenting, to the living organism (user), the measurement data that indicates “Abnormal” as shown in FIG. 5(B) and that has been normally received by the external device 7 and transmitted from the pulse meter 1 with the increased transmission electric power. The measurement data is presented by displaying the measurement data on the screen of the external device 7 so as to draw the living organism's attention.

In this example of presentation, when the living organism selects “CONFIRM” displayed on the lower left portion of the screen, the display of the measurement data is terminated and the measurement data is stored in the external device 7. On the other hand, when the living organism selects “EMERGENCY CONTACT” displayed on the lower right portion of the screen, the external device 7 transfers the measurement data to a predetermined destination such as a host server. In addition, the external device 7 makes an emergency contact with a predetermined emergency contact person/place set in advance in the external device 7, via telephone call or e-mail transmission. Examples of the emergency contact person/place may be the living organism's family member, medical institution, service institution, and the like. The emergency contact may be made from the host server or the like to the predetermined emergency contact person/place.

After carrying out such processing in S17 shown in FIG. 2, the sequence goes back to S10. Specifically, the sequence goes back to the initial state as with the above case. S18 is a first step performed in the case of emergency but in the case of small battery amount. In S18, as shown in FIG. 1, the transmission processing determining section 39 causes the communication section 5 to transmit, to the external device 7, a “transmission request command” (communication request) for use in notifying the external device 7 that the measurement data is to be transmitted thereto.

For the transmission of the transmission request command, the transmission processing determining section 39 instructs the transmission electric power adjusting section 37 to determine transmission electric power. In accordance with the determination of the transmission electric power adjusting section 37, the transmission processing determining section 39 causes the communication section 5 to transmit the transmission request command with the transmission electric power increased. In accordance with the instruction from the transmission processing determining section 39, the communication section 5 transmits the “transmission request command” thereto with the increased transmission electric power. The meaning of the description “with the transmission electric power increased” is the same as that in the aforementioned explanation for S17 shown in FIG. 2, so that explanation therefor is omitted.

Further, processing carried out in cases where the transmission of the “transmission request command” has failed, (e.g., where the pulse meter 1 cannot receives a response sent from that external device 7 and indicating that reception has been made) is the same as that in S17 described above. Specifically, the transmission processing determining section 39 causes the communication section 5 to retransmit the transmission request command to the external device 7 with the increased transmission electric power maintained. In cases where the retransmission of the transmission request command has failed despite the predetermined retransmission processing, it is preferable that the failure of the retransmission be notified to the living organism in the same manner as that in S17.

S19 shown in FIG. 2 is a step of detecting, by way of the “transmission request command” transmitted in S18, that transmission from the pulse meter 1 to the external device 7 can be attained. This step can be realized in many ways. The following explains representative examples thereof.

As one example, the external device 7 shown in FIG. 1 receives the “transmission request command”, and notifies the living organism to carry out an operation of receiving the measurement data, by using, e.g., display on the screen of the external device 7, sound, and vibration. This allows the living organism to recognize that communication for receiving the measurement data needs to be carried out.

Thereafter, the living organism puts the external device 7 sufficiently close to the pulse meter 1 and operates to instruct the external device 7 to start communication with the pulse meter 1. The pulse meter 1 is configured such that the communication condition detecting section 38 receives a “transmission permission command” (communication permission) from the external device 7 as a result of the operation. Hence, the communication condition detecting section 38 receives the transmission permission command from the external device 7 via the communication section 5, thus detecting that the transmission of the measurement data can be attained.

As an alternative way, an input section (not shown) for inputting the living organism's instruction through button operations is provided in the pulse meter 1, and the external device 7 notifies, in the same manner as that in the foregoing example, the living organism that communication for receiving the measurement data needs to be carried out.

As a result of the notification, the living organism puts the external device 7 sufficiently close to the pulse meter 1 and operates the input section of the pulse meter 1 so as to instruct start of communication therebetween. In this way, the communication condition detecting section 38 detects that transmission can be attained. The external device 7 and the pulse meter 1 may be configured as such.

In the above two examples, the living organism carries out the operation for starting communication; however, there is a way by which the living organism (user) never carries out such an operation.

For example, first, reception sensitivity is detected when the external device 7 receives the “transmission request command”. In cases where the external device 7 automatically recognizes, as a result of the detection, that the external device 7 can receive the measurement data from the pulse meter 1 even if the measurement data is transmitted thereto with the “reduced” transmission electric power, the external device 7 automatically transmits the “transmission permission command” to the pulse meter 1. The communication condition detecting section 38 receives the “transmission permission command” from the external device 7 via the communication section 5, thus detecting that transmission can be attained.

As shown in FIG. 2, S20 is a transmission processing step performed in cases where the remaining battery amount is not more than the predetermined amount and in the case of emergency. The transmission processing determining section 39 causes the communication section 5 to transmit, to the external device 7, the measurement data that has been temporarily stored and accumulated in S11 in the temporary memory section 41 shown in FIG. 1 and that indicates the pulse rates (vital information).

For the transmission, the transmission processing determining section 39 shown in FIG. 1 instructs the transmission electric power adjusting section 37 to determine transmission electric power. In accordance with the determination of the transmission electric power adjusting section 37, the transmission processing determining section 39 instructs the communication section 5 to transmit the measurement data with the transmission electric power reduced. The meaning of the description “with the transmission electric power reduced” is the same as that in the case of S15 shown in FIG. 2, so that explanation therefor is omitted.

In cases where the transmission of the measurement data has failed, the transmission processing determining section 39 causes the communication section 5 to retransmit the transmission data to the external device 7 with the reduced transmission electric power maintained.

In cases where the retransmission has failed despite such predetermined retransmission processing, it is preferable to notify the failure of the retransmission to the living organism as is the case with S17 shown in FIG. 2. After the processing in S20, the sequence goes back to S10. Specifically, the sequence goes back to the initial state as with the above case.

By repeating the above processing, the pulse meter 1, put on the finger of the living organism (human organism), normally transmits the measurement data (pulse rates) to the external device 7 (data collecting device, e.g., mobile phone) at the predetermined timing with the reduced transmission electric power.

Further, in cases where an emergency condition is detected, the pulse meter 1 immediately increases the transmission electric power and transmits the measurement data with the increased transmission electric power. A specific example of the case where such an emergency condition is detected is: (i) a case where there is abnormality in the measurement data; (ii) a case where the remaining memory amount is small; or the like. In the case of emergency and a small remaining battery amount, only the transmission request is transmitted with the increased transmission electric power, but the measurement data is transmitted with the reduced transmission electric power.

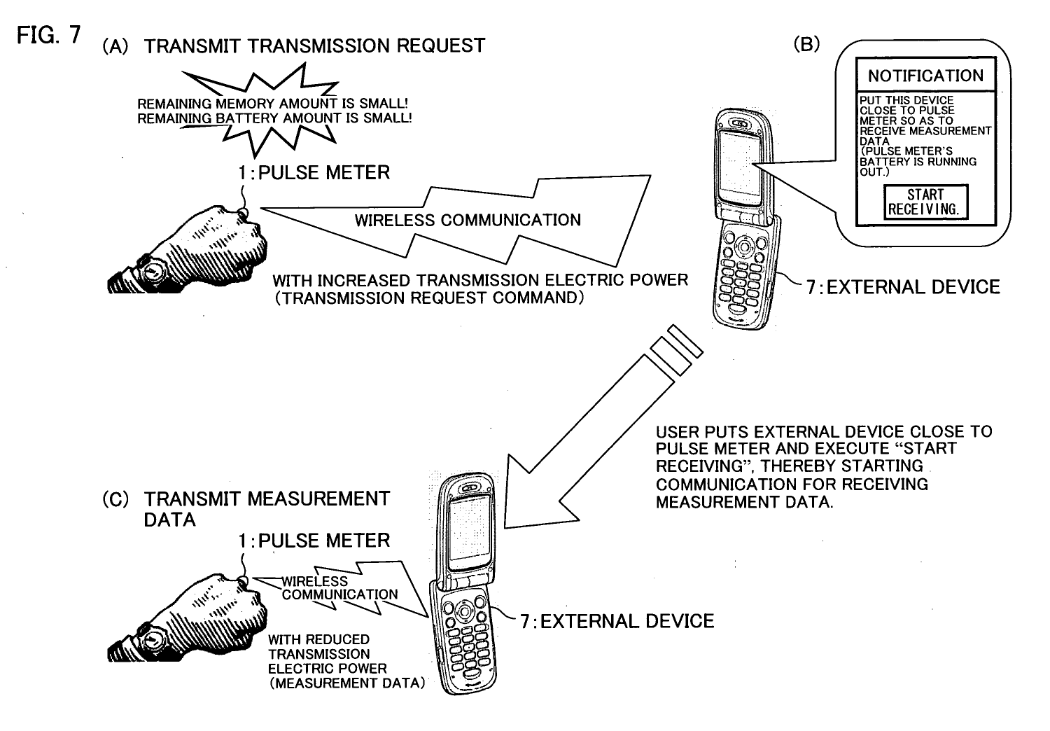

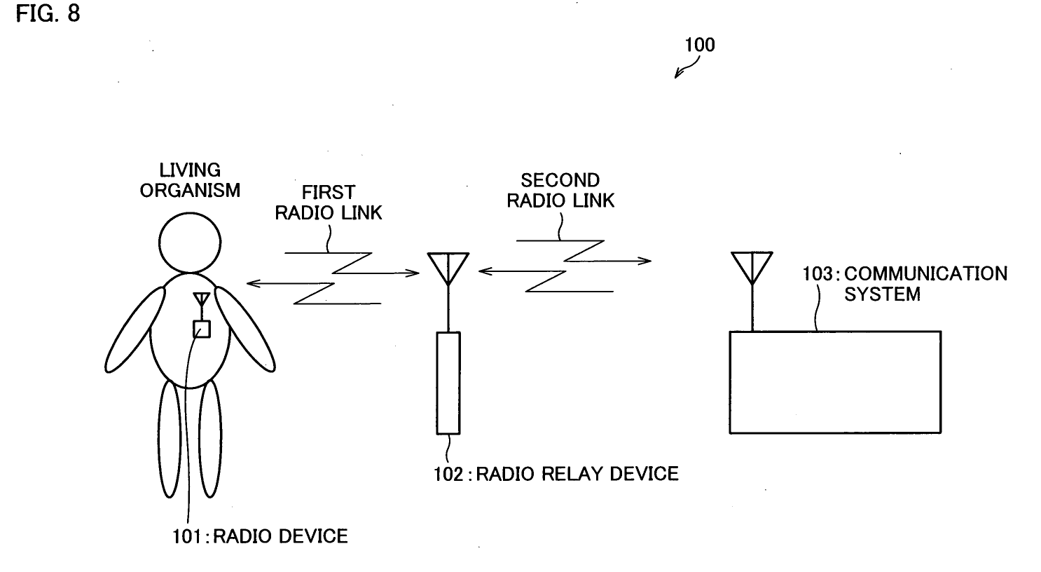

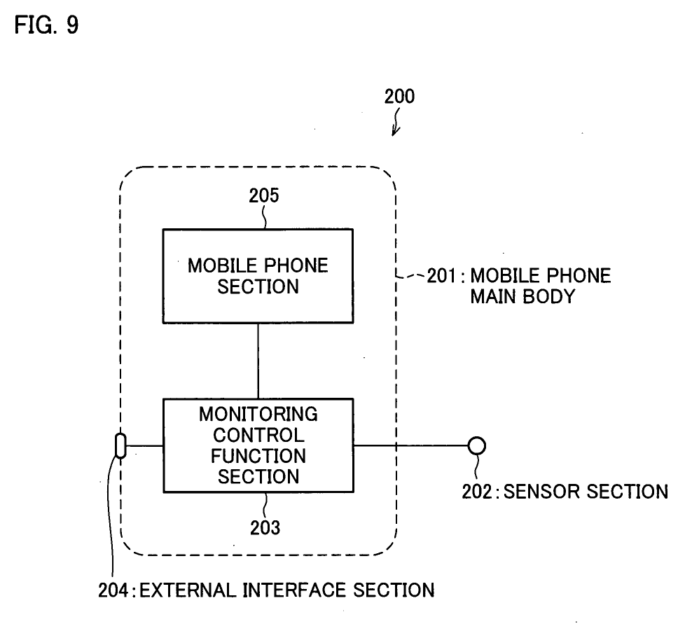

Each of Part (A) of FIG. 7 to Part (C) of FIG. 7 schematically illustrates an example regarding how the living organism operates in cases where the remaining memory amount is judged to be not more than the predetermined amount and the remaining amount of the battery 6 is judged to be not more than the predetermined amount. Part (A) of FIG. 7 shows an example in which the pulse meter 1 transmits the “transmission request command” to the external device 7 with the increased transmission electric power. Part (B) of FIG. 7 shows an example in which the external device 7 normally receives the “transmission request command” and displays it on the screen of the external device 7 so as to urge the living organism to carry out the operation for receiving the measurement data.

See Part (B) of FIG. 7. In this example, the living organism selects “START RECEIVING” displayed on the lower portion of the screen, thereby causing the external device 7 to start the processing of receiving the measurement data. Before starting the operation of selecting “START RECEIVING”, the living organism puts the external device 7 close to the pulse meter 1 up to such a distance that the external device 7 can receive the measurement data even when the pulse meter 1 transmits it with the reduced transmission electric power. Part (C) of FIG. 7 shows an example in which: in response to the living organism's operation of selecting “START RECEIVING” displayed on the external device 7 thus put close to the pulse meter 1, the pulse meter 1 transmits the measurement data thereto with the reduced transmission electric power.

As described above, when the living organism is not in an abnormal condition, the vital information measuring device of the present embodiment carries out measurement less frequently and accumulates the measurement data in the memory such that wireless communication is carried out as little times as possible. Moreover, the measurement data is transmitted to the external device (data collecting device) at the predetermined timing with the reduced transmission electric power, with the result that electric power consumption is restrained as much as possible. This allows downsizing and weight saving of the device in connection with the battery capacity and allows improvement of the life of the battery.

In the meanwhile, in cases where an emergency condition is detected, e.g., where there is abnormality in the measurement data, where the remaining memory amount is small, or the like, the transmission electric power is increased immediately and the measurement data is transmitted therewith. This makes it possible to transmit the measurement data immediately with reliability of the wireless communication improved as much as possible.

Further, only the transmission request is transmitted with the increased transmission electric power, whereas the measurement data is transmitted with the reduced transmission electric power. This makes it possible to restrain the electric power consumption and transmit the measurement data immediately even when the remaining battery amount is small. Further, in the case of emergency but a small remaining battery amount, the transmission electric power may be increased only for the transmission of the transmission request but may be reduced for the transmission of the measurement data. Examples of the case of emergency include: a case where there is abnormality in the measurement data; a case where the remaining memory amount is small; and the like.

The present embodiment exemplifies the ring type pulse meter put on the finger of the human organism. However, the technical scope of the present invention encompasses various vital information measuring devices, each of which is put on a certain part of the living organism so as to measure the vital information and transmits it via wireless or wired communication. Examples of the various vital information measuring device include: an oxygen saturation meter, a blood pressure meter, an electrocardiograph, a thermometer, a passometer, and a device for measuring a physical activity in accordance with acceleration, angular velocity, and/or the like. Further, the examinee subjected to the measurement is not limited to a human organism, but may be any organism other than a human being.

In the above explanation, a mobile phone is exemplified as the external device 7, which receives the vital information; however, the present invention is not limited to this. For example, various data collecting devices may be used as the external device 7, such as data collecting devices installed in a house, a working place, a public facility, and the like. Specific examples of the data collecting devices are a PC (personal computer) capable of carrying out communication via a wireless access point or via a wireless LAN (local area network); other wireless gateway devices; and the like.

Finally, the respective blocks of the pulse meter 1, especially the electric power consumption control section 3, may be constituted by hardware logic, or may be realized by software with the use of a CPU as follows.

In this case, the pulse meter 1 includes (i) a CPU (central processing unit) for executing instructions of a control program realizing each function; (ii) a ROM (read only memory) storing the above program; (iii) a RAM (random access memory) for expanding the program; (iv) a storage device (storage medium), such as a memory, storing the program and various types of data; and the like. Therefore, the object of the present invention is achieved by: (i) providing, in the pulse meter 1, a storage medium which stores a computer-readable program code (executable program, intermediate code program, a source program) of the control program of the pulse meter 1 that is software for realizing the function, and (ii) causing a computer (CPU, or MPU) to read out and execute the program code stored in the storage medium.

Examples of the storage medium are: tapes such as a magnetic tape and a cassette tape; magnetic disks such as a floppy® disk and a hard disk; disks such as a CD-ROM (compact disk read only memory), a magnetic optical disk (MO), a mini disk (MD), a digital video disk (DVD), and a CD-Recordable (CD-R); and the like. Further, the storage medium may be: a card such as an IC card or an optical card; or a semiconductor memory such as a mask ROM, an EPROM (electrically programmable read only memory), an EEPROM (electrically erasable programmable read only memory), or a flash ROM.

Further, the pulse meter 1 may be so configured as to be connectable to a communication network, and the program code may be supplied to the pulse meter 1 via the network. The communication network is not particularly limited. Specific examples thereof are: the Internet, intranet, extranet, LAN (local area network), ISDN (integrated services digital network), VAN (value added network), CATV (cable TV) communication network, virtual private network, telephone network, mobile communication network, satellite communication network, and the like. Further, a transmission medium (channel) constituting the communication network is not particularly limited. Specific examples thereof are: (i) a wired channel using an IEEE1394, a USB (universal serial bus), a power-line communication, a cable TV line, a telephone line, a ADSL line, or the like; or (ii) a wireless channel using IrDA, infrared rays used for a remote controller, Bluetooth®, IEEE802.11, HDR (High Data Rate), a mobile phone network, a satellite connection, a terrestrial digital network, or the like. Note that the present invention can be realized by a form of a computer data signal (a series of data signals) embedded in a carrier wave realized by electronic transmission of the program code.

In addition to the above configuration, the vital information measuring device of the present invention may be configured such that: the electric power consumption control means includes communication condition adjusting means for adjusting a communication condition of the communication means, and the electric power consumption control means controls the electric power consumption of the vital information measuring device by causing the communication condition adjusting means to adjust the communication condition of the communication means in accordance with the result of the judgment carried out by the vital condition judging means.

According to the above configuration, the communication condition adjusting means determines transmission electric power, a transmission processing method, and the like in accordance with the result of the detection carried out by the vital condition judging means and the communication condition between the vital information measuring device and the external device, and instructs the communication means to carry out wireless or wired communication. Moreover, the electric power consumption control means causes the communication condition adjusting means to adjust the communication condition of the communication means, thereby controlling the electric power consumption of the vital information measuring device.

This makes it possible to provide such a vital information measuring device that is downsized, has a light weight, and allows improvement of battery life, by reducing, as much as possible, electric power consumption required for wireless or wired communication and processing for the communication, and that is capable of immediately transmitting measurement data in the case of emergency with reliability of the wireless or wired communication improved as much as possible. A specific example of the case of emergency is a case where there is detected abnormality in the living organism; or the like.

Further, in addition to the above configuration, the vital information measuring device of the present invention may be arranged such that: the electric power consumption control means includes measurement frequency adjusting means for adjusting how frequent the measuring means carries out the measurement, and the electric power consumption control means controls the electric power consumption of the vital information measuring device by causing the measurement frequency adjusting means to adjust, in accordance with the result of the judgment carried out by the vital condition judging means, how frequent the measuring means carries out the measurement.

According to the above configuration, the electric power consumption control means causes the measurement frequency adjusting means to adjust how frequent the measuring means carries out the measurement, thereby controlling electric power consumption of the vital information measuring device.

This makes it possible to provide such a vital information measuring device that is downsized, has light weight, and allows improvement of battery life, by reducing, as much as possible, electric power consumption required for measurement of vital information, and that is capable of adjusting, in the case of emergency, how frequent the measurement is carried out. A specific example of the case of emergency is a case where there is detected abnormality in the living organism; or the like.

In addition to the above configuration, the vital information measuring device according to the present invention may further include: device condition judging means for judging a condition of the vital information measuring device, wherein: the electric power consumption control means controls the electric power consumption of the vital information measuring device in accordance with a result of the judgment carried out by the device condition judging means.

According to the above configuration, the vital information measuring device further includes the device condition judging means for judging the condition of the vital information measuring device, and the electric power consumption control means controls the electric power consumption of the vital information measuring device in accordance with the result of the judgment carried out by the device condition judging means.

This makes it possible to provide such a vital information measuring device that is downsized, has light weight, and allows improvement of battery life, by reducing, as much as possible, electric power consumption required for measurement of vital information, wireless or wired communication, and processing for the communication, and that is capable of immediately transmitting measurement data in the case of emergency with reliability of the wireless or wired communication improved as much as possible. A specific example of the case of emergency is a case where there is detected abnormality in the living organism and/or the vital information measuring device; or the like.

In addition to the above configuration, the vital information measuring device according to the present invention may be arranged such that: the communication condition adjusting means includes transmission timing adjusting means for adjusting a timing at which the communication means transmits, to the external device, the vital information measured by the measuring means, so as to adjust the communication condition of the communication means.

According to the above configuration, the transmission timing adjusting means adjusts a timing at which the communication means transmits, to the external device, the vital information measured by the measuring means.

Here, the phrase “adjust the timing at which the communication means transmits the vital information” encompasses not only (i) a case of adjusting the timing in accordance with a condition of time information, such as a constant cycle or a fixed time, (ii) a case of adjusting the timing in accordance with a condition other than the time information, and (iii) a case of adjusting the timing in accordance with a combination of the above conditions.

For example, the timing is adjusted adequately such that: measurement data are accumulated until a specific data transmission timing comes and the measurement data thus accumulated are transmitted to the external device all together when the specific data transmission timing has come.

An alternative example is as follows. That is, in cases where processing of retransmitting the measurement data is set to be carried out but the results of the judgments carried out by the vital condition judging means and the device condition judging means indicate that urgent transmission thereof is not required, the measurement data is not immediately retransmitted at the current transmission timing but is going to be transmitted at the next transmission timing together with measurement data to be obtained next time.

In addition to the above configuration, the vital information measuring device of the present invention may be configured such that: the communication condition adjusting means includes transmission electric power adjusting means for adjusting transmission electric power with which the communication means transmits, to the external device, the vital information measured by the measuring means, so as to adjust the communication condition of the communication means.

According to the above configuration, the transmission electric power adjusting means arbitrarily adjusts the strength of the transmission electric power with which the communication means carries out data transmission.

Such a function of the transmission electric power adjusting means allows the electric power consumption control means to control the electric power consumption of the vital information measuring device.

In addition to the above configuration, the vital information measuring device of the present invention may be configured such that: in accordance with the result of the judgment carried out by the device condition judging means, the transmission electric power adjusting means adjusts the transmission electric power, used by the communication means, to a first transmission electric power for a purpose of transmission of a communication request to the external device, and the transmission electric power adjusting means adjusts the transmission electric power, used by the communication means, to a second transmission electric power when a communication permission replying to the communication request is detected.

According to the above configuration, the transmission electric power used by the communication means is arbitrarily settable at the first transmission electric power and the second transmission electric power.

By appropriately adjusting the transmission electric power at the first transmission electric power and the second transmission electric power, it is possible to control the electric power consumption of the communication means.

Here, the following explains the “first transmission electric power” and the “second transmission electric power”. For ease of explanation, “normal electric power” is defined first. The “normal electric power” refers to transmission electric power falling with in a range from the maximal transmission electric power to the minimal electric power each allowing for communication between the vital information measuring device and the external device separated from each other by a physical distance assumed in normal use (distance assumed to be maximal, distance assumed to be minimal); or (ii) transmission electric power close to the minimal normal electric power. The same explanation will not be repeated below.

In cases where “the first transmission electric power” is the maximal “normal electric power” or transmission electric power close to the maximal normal electric power, the “second transmission electric power” is set at the minimal “normal electric power” or transmission electric power close to the minimal normal electric power. On the other hand, in cases where the “second transmission electric power” is the maximal “normal electric power” or transmission electric power close to the maximal normal electric power, the “first transmission electric power” is set at the minimal “normal electric power” and transmission electric power close to the minimal “normal electric power”. As such, the transmission electric power adjusting means is configured to set the transmission electric power selectively at (i) the maximal “normal electric power” or the transmission electric power close to the maximal normal electric power and (ii) the minimal “normal electric power” or the transmission electric power close to the minimal normal electric power, with the result that an average of electric power consumption of the vital information measuring device per unit time is reduced.

Note that the transmission electric power “close to the maximal normal electric power” refers to transmission electric power larger than the average value of the maximal transmission electric power and the minimal transmission electric power.

Note also that the transmission electric power “close to the minimal normal electric power” refers to transmission electric power smaller than the average value of the maximal transmission electric power and the minimal transmission electric power.

Here, the following explains a relation between (i) the “communication request” and the “communication permission” and (ii) the “first transmission electric power” and the “second transmission electric power”. The description herein assumes the case of emergency but a small remaining electric power amount (case where it is judged that there is abnormality in the living organism and the device); however, the present invention is not limited to this. In such a case, the communication condition adjusting means causes the communication means to transmit, to the external device, the “communication request”, which is for use in notifying the external device that the measurement data is to be transmitted.

For the transmission of the communication request, in accordance with the determination of the transmission electric power adjusting means, the communication condition adjusting means causes the communication means to transmit the transmission request command with the “normal maximal electric power” or the transmission electric power closed to the maximal normal electric power (first transmission electric power). Likewise, in cases where the transmission of the “communication request” has failed, (e.g., where the vital information measuring device cannot receives a response sent from that external device and indicating that reception has been made), the communication condition adjusting means causes the communication section to retransmit the communication request to the external device with the increased transmission electric power maintained (with the first transmission electric power).

Next, the external device detects reception sensitivity when the external device receives the “transmission request command”. In cases where the external device automatically recognizes, as a result of the detection, that the external device can receive the measurement data from the vital information measuring device even if the measurement data is transmitted thereto with the transmission electric power reduced to be smaller than the “normal electric power” (with the second transmission electric power), the external device automatically transmits the “communication permission” to the vital information measuring device. The communication condition adjusting means receives the “communication permission” from the external device via the communication means, thus detecting that transmission can be attained.

The description herein exemplifies the case where the “communication permission” is received from the external device; however, the present invention is not limited to this. The communication permission may be detected when the user instructs the vital information measuring device to start communication.

In addition to the above configuration, the vital information measuring device may further include: memory means for storing the vital information, wherein: the device condition judging means includes remaining memory amount detecting means for detecting a remaining memory amount of the memory means, and the device condition judging means judges the condition of the vital information measuring device in accordance with a result of the detection carried out by the remaining memory amount detecting means.

According to the above configuration, the device condition judging means is capable of detecting the remaining memory amount of the memory means.

This allows the device condition judging means to judge the condition of the vital information measuring device in accordance with the remaining memory amount of the memory means.

Hence, in cases where the device condition judging means judges, in accordance with the result of the detection carried out by the remaining memory amount detecting means, that there is left only a small memory amount in the memory means for example, the transmission timing adjusting means set, at an earlier timing, the data transmission timing at which the communication means carries out data transmission, or the measurement frequency adjusting means causes the measuring means to carry out the measurement less frequently. This prevents shortage of the memory amount of the memory means of the vital information measuring device.

In addition to the above configuration, the vital information measuring device of the present invention may further include: a power source for supplying electric power to the vital information measuring device, wherein: the device condition judging means includes electric power remaining amount detecting means for detecting a remaining amount of electric power that is to be supplied from the power source, and the device condition judging means judges the condition of the vital information measuring device in accordance with a result of the detection carried out by the electric power remaining amount detecting means.

According to the above configuration, the device condition judging means is capable of detecting the remaining amount of electric power that is to be supplied from the power source.

This allows the device condition judging means to judge the condition of the vital information measuring device in accordance with the remaining amount of electric power that is to be supplied from the power source.

Hence, in cases where the device condition judging means judges, in accordance with the result of the detection carried out by the electric power remaining amount detecting means, that there is left a small amount of electric power to be supplied from the power source for example, the transmission timing adjusting means delays the data transmission timing at which the communication means carries out data transmission, the transmission electric power adjusting means reduces the transmission electric power to be used by the communication means, to transmission electric power smaller than the “normal electric power”, and the measurement frequency adjusting means causes the measuring means to carry out the measurement less frequently. This prevents shortage of the electric power to be supplied from the power source vital information measuring device.

Further, in addition to the above configuration, the vital information measuring device of the present invention may be configured such that: the vital evaluation information includes an evaluation threshold value for evaluating the vital information measured by the measuring means.

According to the above configuration, the vital condition judging means compares (i) the vital information measured by the measuring means with (ii) the threshold value (evaluation threshold value) for judging whether or not the condition of the living organism is abnormal, thereby judging the condition thereof.

Here, the following explains the “evaluation threshold value”. The “evaluation threshold value” is a vital information value, based on which whether the living organism is in a normal condition or an abnormal condition is judged. Examples of an evaluation threshold value for pulse rate include: the normal lower limit value, the middle value, and the normal upper limit value of pulse rate for each age and sex. Reference examples of an evaluation threshold value for items other than pulse rate includes: various evaluation threshold values for use in judging the condition of the living organism, such as blood pressure value, arterial blood, oxygen saturation, body temperature, measurement value of electrocardiograph.

As such, the vital condition judging means of the vital information measuring device judges whether or not the living organism is in an emergency condition or not, by using such an easy way, i.e., by comparing the measured vital information with the evaluation threshold value. Hence, the vital information measuring device is capable of notifying the user that he/she is in an emergency condition.

In addition to the above configuration, the vital information measuring device according to the present invention may be configured such that: the vital evaluation information includes vital reference information, which is information referred together with the evaluation threshold value, and the vital reference information is information regarding an attribute of the living organism, information regarding a daily physical condition of the living organism, or information regarding health management of the living organism.

According to the above configuration, the vital condition judging means compares (i) the vital information measured by the measuring means with (ii) the combination of the evaluation threshold value and the vital reference information allowing for more accurate judgment, thereby judging the condition of the living organism.

Thus, the vital condition judging means is allowed to judge the condition of the living organism more accurately, thereby restraining unnecessary electric power consumption of the vital information measuring device because of mistakenly judging that the living organism is in an emergency condition. Also, notifications to the living organism are not made excessively and more than necessary.

Here, the following explains the “vital reference information”. The “vital reference information” is information regarding the living organism's attribute, physical condition under normal circumstances, or health management, and is information referred together with the evaluation threshold value. As described below, examples of the “vital reference information” include: various information regarding the physical body of the living organism and health thereof, such as the living organism's “basic information”, “physical information”, and “medical history-health information”.

The basic information does not have a direct relation with the living organism's health condition, but is information representing attributes intrinsic to the living organism. Examples of the “basic information” regarding the living organism include name, sex, age, and date of birth. On the other hand, the “physical information” is information that is based on information measured from the physical body of the living organism and that indicates the living organism's recent physical condition. Examples of the “physical information” regarding the living organism include the living organism's height, weight, pulse rate (average range), maximal blood pressure (average value), minimal blood pressure (average value), oxygen saturation (average value), body temperature when awaking (average value), and body temperature when sleeping (average value).

The “medical history-health information” regarding the living organism is information regarding the living organism's health management, such as (i) medical history regarding diseases and symptoms that the living organism is suffering at present and has suffered in past and (ii) supplementary information regarding the living organism's health condition (e.g., knowledge from the medical institute, etc., to which the living organism goes).

Apart from these examples, there may be provided various vital reference information regarding the physical body and health of the user (living organism) and regarding the physical body and health of a living organism other than the user, such as (i) the user's parents, grandparents, children, and relatives who are quite similar to the user physically and (ii) a living organism having physical characteristics and constitution similar to the user. This makes it possible for the vital condition judging means to make a judgment finely.

Note that the vital information measuring device may be realized by a computer. In this case, the present invention encompasses (i) the vital information measuring device's control program for realizing the vital information measuring device by a computer by causing the computer to operate as the respective means; and (ii) a computer-readable storage medium storing the control program.

The present invention is not limited to the description of the embodiments above, but may be altered by a skilled person within the scope of the claims. An embodiment based on a proper combination of technical means disclosed in different embodiments is encompassed in the technical scope of the present invention.

The embodiments and concrete examples of implementation discussed in the foregoing detailed explanation serve solely to illustrate the technical details of the present invention, which should not be narrowly interpreted within the limits of such embodiments and concrete examples, but rather may be applied in many variations within the spirit of the present invention, provided such variations do not exceed the scope of the patent claims set forth below.

What is claimed is:

1. A vital information measuring device, comprising:
communication means for communicating with an external device;
measuring means for measuring vital information of a living organism;
vital condition judging means for judging a condition of the living organism by comparing (i) the vital information measured by the measuring means with (ii) vital evaluation information for evaluating the vital information; and
electric power consumption control means for controlling electric power consumption of the vital information measuring device in accordance with a result of the judgment carried out by the vital condition judging means.

2. The vital information measuring device as set forth in claim 1, wherein:
the electric power consumption control means includes communication condition adjusting means for adjusting a communication condition of the communication means, and
the electric power consumption control means controls the electric power consumption of the vital information measuring device by causing the communication condition adjusting means to adjust the communication condition of the communication means in accordance with the result of the judgment carried out by the vital condition judging means.

3. The vital information measuring device as set forth in claim 1, wherein:
the electric power consumption control means includes measurement frequency adjusting means for adjusting how frequent the measuring means carries out the measurement, and
the electric power consumption control means controls the electric power consumption of the vital information measuring device by causing the measurement frequency adjusting means to adjust, in accordance with the result of the judgment carried out by the vital condition judging means, how frequent the measuring means carries out the measurement.

4. The vital information measuring device as set forth in claim 1, further comprising:
device condition judging means for judging a condition of the vital information measuring device,
wherein:
the electric power consumption control means controls the electric power consumption of the vital information measuring device in accordance with a result of the judgment carried out by the device condition judging means.

5. The vital information measuring device as set forth in claim 2, wherein:
the communication condition adjusting means includes transmission timing adjusting means for adjusting a timing at which the communication means transmits, to the external device, the vital information measured by the measuring means, so as to adjust the communication condition of the communication means.

6. The vital information measuring device as set forth in claim 2, wherein:
the communication condition adjusting means includes transmission electric power adjusting means for adjusting transmission electric power with which the communication means transmits, to the external device, the vital information measured by the measuring means, so as to adjust the communication condition of the communication means.

7. The vital information measuring device as set forth in claim 6, wherein:
in accordance with the result of the judgment carried out by the device condition judging means, the transmission electric power adjusting means adjusts the transmission electric power, used by the communication means, to a first transmission electric power for a purpose of transmission of a communication request to the external device, and
the transmission electric power adjusting means adjusts the transmission electric power, used by the communication means, to a second transmission electric power when a communication permission replying to the communication request is detected.

8. The vital information measuring device as set forth in claim 4, further comprising:
memory means for storing the vital information,
wherein:
the device condition judging means includes remaining memory amount detecting means for detecting a remaining memory amount of the memory means, and
the device condition judging means judges the condition of the vital information measuring device in accordance with a result of the detection carried out by the remaining memory amount detecting means.

9. The vital information measuring device as set forth in claim 4, further comprising:
a power source for supplying electric power to the vital information measuring device,
wherein:
the device condition judging means includes electric power remaining amount detecting means for detecting a remaining amount of electric power that is to be supplied from the power source, and
the device condition judging means judges the condition of the vital information measuring device in accordance with a result of the detection carried out by the electric power remaining amount detecting means.

10. The vital information measuring device as set forth in claim 1, wherein:
the vital evaluation information includes an evaluation threshold value for evaluating the vital information measured by the measuring means.

11. The vital information measuring device as set forth in claim 10, wherein:
the vital evaluation information includes vital reference information, which is information referred together with the evaluation threshold value, and
the vital reference information is information regarding an attribute of the living organism, information regarding a daily physical condition of the living organism, or information regarding health management of the living organism.

12. An electric power consumption control method, comprising:
a measuring step in which measuring means of a vital information measuring device measures vital information of a living organism;
a vital condition judging step in which vital condition judging means of the vital information measuring device judges a condition of the living organism by comparing (i) the vital information measured in the measuring step with (ii) vital evaluation information for evaluating the vital information; and
an electric power consumption control step in which electric power consumption control means of the vital information measuring device controls electric power consumption of the vital information measuring device in accordance with a result of the judgment carried out in the vital condition judging step.

13. A vital information measuring program for causing a computer to operate as the respective means of the vital information measuring device as set forth in claim 1.

14. A computer-readable storage medium storing the vital information measuring program as set forth in claim 13.
